(19) United States
(12) Patent Application Publication
(10) Pub. No.: US 2022/0356226 A1
(43) Pub. Date: Nov. 10, 2022

(54) COMPOUNDS AND METHODS FOR THE TREATMENT OF ALZHEIMER'S DISEASE

(71) Applicant: UNIVERSIDAD DEL PAÍS VASCO / EUSKAL HERRIKO UNIBERTSITATEA, Leioa (ES)

(72) Inventors: José Luis ZUGAZA, Leioa (ES); Francisco LLAVERO, Leioa (ES); Miriam LUQUE, Leioa (ES); Alazne ARRAZOLA, Leioa (ES); Carolina ORTIZ, Leioa (ES); Tania QUINTELA, Leioa (ES); Anne WYSSENBACH, Leioa (ES); Carlos MATUTE ALMAU, Leioa (ES); Elena ALBERDI ALFONSO, Leioa (ES)

(21) Appl. No.: 17/765,137

(22) PCT Filed: Oct. 23, 2020
(86) PCT No.: PCT/EP2020/079885

§ 371 Date: Mar. 30, 2022

(30) Foreign Application Priority Data
Oct. 24, 2019 (ES) P201930939

(51) Int. Cl. C07K 14/705; A61P 25/28; A61K 31/445; A61K 31/325; A61K 31/55; A61K 31/13; A61K 39/395; A61K 31/145; A61K 31/047

(52) U.S. Cl. CPC C07K 14/7055; A61P 25/28; A61K 31/445; A61K 31/325; A61K 31/55; A61K 31/13; A61K 39/3955; A61K 31/145; A61K 31/047; A61K 38/00

(57) ABSTRACT

The present invention relates to the development of polypeptides useful for the treatment of diseases associated with amyloid deposits, and more specifically for the treatment of Alzheimer's disease. The invention also relates to compositions comprising the developed polypeptides and to a method for the identification of compounds useful for the treatment of diseases associated with the formation of amyloid deposits.

### FIELD OF THE INVENTION

The invention is comprised in the field of diseases caused by amyloid deposit accumulation, more specifically in the field of new therapeutic target identification and new therapy development for these diseases.

### BACKGROUND OF THE INVENTION

Amyloidosis is a generic term, used to refer to a group of diseases of diverse etiology and variable prognosis and treatment, with a common characteristic: all of them are caused by the extracellular deposition of an insoluble, proteolysis-resistant, protein material, referred to as amyloid material. One of the many peptides appearing in amyloid accumulations or deposits is the beta-amyloid (β-amyloid, AB, or Aβ) peptide originating from the amyloid precursor protein (APP).

In vitro studies indicate that APP (sAPPα) processing through the non-amyloidogenic pathway can act as an autocrine signal to stimulate cell proliferation and adhesion and support nerve growth in PC12 cells. However, if the APP is processed through the amyloidogenic pathway, a peptide having 39-43 amino acids, specifically beta-amyloid peptide (β-amyloid peptide or Aβ peptide) which has been indicated as the primary neurotoxic factor in the pathogenesis of neurodegenerative processes, e.g., Alzheimer's, is generated. Said peptide is toxic to endothelial cells, smooth muscle cells, astrocytes, neurons, and oligodendrocytes in vitro. The mechanisms through which the Aβ peptide exerts its cytotoxic action are not defined, although it would appear that oxidative processes may be involved in the generation of this toxicity, inducing cell death.

The presence of Aβ peptide deposits in the brain has been linked to various diseases, such as Alzheimer's disease (AD), memory loss, attention deficit symptoms associated with Alzheimer's disease, diffuse Lewy body-type Alzheimer's disease, mild cognitive impairment, hereditary cerebral hemorrhage with amyloidosis-Dutch type, β-amyloid angiopathy, and cerebral hemorrhage such as cerebral hemorrhage due to solitary cerebral amyloid angiopathy, prion infections, type II diabetes, degenerative dementias, including dementias of mixed degenerative and vascular origin, frontotemporal dementia, presenile dementia, senile dementia, dementia associated with acquired immunodeficiency syndrome (AIDS), parkinsonian disorders such as Parkinson's disease (PD), subacute sclerosing panencephalitis parkinsonism, post-encephalitic parkinsonism, pugilistic encephalitis, Guam parkinsonism-dementia complex, Pick's disease, multiple system atrophy (MSA), progressive supranuclear paralysis (PSP), corticobasal degeneration (CBD), Down syndrome, Lewy body disease, Huntington's disease, amyotrophic lateral sclerosis, multiple sclerosis, and neurotraumatic diseases such as acute stroke, epilepsy, mood disorders such as depression, schizophrenia and bipolar disorders, ischemia, brain injury, particularly traumatic brain injury, inflammation, and chronic inflammatory diseases.

Current efforts focus on discovering possible signaling cascades that may be mediated by the Aβ peptide. There are several signaling pathways that may be involved in cell damage and that appear to be activated by the Aβ peptide. For example, it seems clear that the effects of Aβ peptide are associated with oxidative stress, mitochondrial dysfunction, alteration of Ca²⁺ homeostasis, NO generation, microglia activation, and others. However, the explanation of the causal relation or the exact sequence of these events is still under dispute.

Aβ peptide oligomers and Aβ-derived diffusible ligands (ADDL) in primary neuronal cultures can bind avidly to membrane receptors or specific neuronal plasma membrane regions and induce rapid cell death through the mitochondrial apoptotic pathway. In contrast, Aβ peptide fibrils seem to induce a more chronic form of neuritic dystrophy and neuronal death. The rapid toxic effects of Aβ peptide have been associated with a pro-oxidant effect of the peptide and can be mediated in part through RAGE (receptor for advanced glycation end products). The Aβ peptide can also induce apoptosis through caspase and calpain activation. Another mechanism of toxicity may involve aberrant activation of cell cycle re-entry in neurons, which has been observed in neuronal cultures treated with Aβ peptide and in AD. Little is known about the factors regulating the generation of toxic Aβ peptide aggregates in the aging brain, although recent studies suggest possible roles for the signaling of insulin-like growth factor 1/insulin (IGF-1) and calcium homeostasis. Another class of Aβ peptide-activated signaling pathways is involved in microglia inflammatory response. Amyloid deposits are closely associated with microglia activation in AD and in APP transgenic mice.

Therefore, due to the participation of Aβ peptide in various diseases, there is a need to identify new targets, as well as compounds acting on said targets, for the purpose of developing therapeutic agents which effectively reduce cell death caused by Aβ peptide deposition.

### SUMMARY OF THE INVENTION

The authors of the present invention have shown that polypeptides containing a region formed by amino acids 1-20 of integrin β1 are capable of binding specifically to amyloid β-protein 1-42 (hereinafter Aβ 1-42) and that said binding prevents the Aβ peptide 1-42 from exerting its toxic action on astrocytes. Specifically, pre-incubating Aβ 1-42 with this polypeptide significantly reduces the capacity of Aβ 1-42 to induce Rac1 activation in astrocytes, to induce the formation of reactive oxygen species in astrocytes, to induce astrogliosis in astrocytes, and to reduce GRP78 expression in the astrocytes found in the brains of mice to which Aβ 1-42 has been administered. Likewise, the polypeptides containing the region formed by amino acids 1-20 of integrin β1 keep cellular integrin β1 free such that it can exert its action physiologically.

Based on these results, the researchers propose that a polypeptide comprising amino acids 1-20 of integrin β1 may be useful for the treatment of diseases characterized by the formation of beta amyloid deposits including, among others, Alzheimer's disease.

Therefore, in a first aspect, the invention relates to a polypeptide comprising sequence SEQ ID NO:1 or a functionally equivalent variant thereof for use in the treatment of a disease associated with the formation of amyloid deposits.

In another aspect, the invention relates to a composition comprising a polypeptide comprising sequence SEQ ID NO:1 or a functionally equivalent variant thereof and a compound suitable for the treatment of a disease associated with the formation of amyloid deposits.

In another aspect, the invention relates to a method for the identification of a compound capable of inhibiting amyloid deposit-induced cell death and/or suitable for the treatment of a disease associated with the formation of amyloid deposits, which method comprises:
a) contacting a first sample of a cell population with the amyloid protein and with a candidate compound and a second sample of said cell population with the amyloid protein and with a polypeptide comprising sequence SEQ ID NO:1 or a functionally equivalent variant thereof; and
b) determining in the cell populations of the first and second samples the level of at least one marker associated with amyloid deposit-induced cell death,

wherein if the level of the at least one marker associated with amyloid deposit-induced cell death in the first sample is lower than the level of said marker in the second sample, it is indicative of the candidate compound being capable of inhibiting amyloid deposit-induced cell death and/or being useful for the treatment of a disease associated with the formation of amyloid deposits.

### BRIEF DESCRIPTION OF THE DRAWINGS

FIG. 1. Depiction of integrin β1 extracellular structure with its corresponding amino acid sequences, depicting recombinant peptides Rs, Rd, Rw, and Rt.

FIG. 2. In vitro binding assay between the recombinant peptides indicated in FIG. 1 and the β amyloid peptide 1-42.

FIG. 3. Action of the effective minimum region Rs on β amyloid peptide 1-42-mediated Rac GTPase activation.

FIG. 4. Action of the effective minimum region Rs on β amyloid peptide 1-42-mediated ROS generation.

FIG. 5A. GST-Rs peptide administration prevents reactive astrogliosis in the dentate gyrus of the brain of mice to which β amyloid peptide 1-42 has been injected. The dot plot shows the quantitative analysis of the marked areas for astrogliosis marker, GFAP.

FIG. 5B. GST-Rs peptide administration prevents reactive astrogliosis in the dentate gyrus of the brain of mice to which β amyloid peptide 1-42 has been injected. The dot plot shows the quantitative analysis of the marked areas for astrogliosis marker, S100 β.

FIG. 5C. Control peptide (GST0) administration does not prevent reactive astrogliosis in the dentate gyrus of the brain of mice to which β amyloid peptide 1-42 has been injected. The dot plot shows the quantitative analysis of the marked areas for astrogliosis marker, GFAP.

FIG. 5D. Control peptide (GST0) administration does not prevent reactive astrogliosis in the dentate gyrus of the brain of mice to which β amyloid peptide 1-42 has been injected. The dot plot shows the quantitative analysis of the marked areas for astrogliosis marker, S100 β.

FIG. 6A. GST-Rs peptide administration reduces GRP78 expression in S100 β positive astrocytes in the dentate gyrus of the brain of mice injected with β amyloid peptide 1-42. Quantitative analysis of GRP78 levels in S100 β-positive astrocytes.

FIG. 6B. Control peptide (GST0) administration does not reduce GRP78 expression in S100 β-positive astrocytes in the dentate gyrus of the brain of mice injected with β amyloid peptide 1-42. Quantitative analysis of GRP78 levels in S100 β-positive astrocytes.

FIG. 7. Working model of the peptides.

### DETAILED DESCRIPTION OF THE INVENTION

Medical Uses of the Invention

In a first aspect, the invention relates to a polypeptide comprising sequence SEQ ID NO:1 or a functionally equivalent variant thereof for use in the treatment of a disease associated with the formation of amyloid deposits.

As it is used herein, the term “polypeptide” refers to any sequence of two or more amino acids linearly linked by amide bonds (peptide bonds) regardless of length, post-translational modification, or function. “Polypeptide,” “peptide,” and “protein” are used interchangeably herein. The term “polypeptide” also refers to the products of post-expression modifications of polypeptide including, among others, glycosylation, acetylation, phosphorylation, amidation, derivatization by protecting/blocking groups, proteolytic cleavage, or modification by non-natural intermediate amino acids. A polypeptide can be derived from a natural biological source or produced by means of recombinant technology, but is not necessarily translated from a nucleic acid. The polypeptides according to the present invention can be produced by means of any method known to the skilled person, including by means of chemical synthesis. Fragments, derivatives, analogs, or variants of the preceding polypeptides, and any combination thereof, are also included as polypeptides of the present invention. These variants can be naturally occurring or non-natural variants. The non-natural variants can be produced using mutagenesis techniques known in the art. The polypeptide variants may comprise conservative or non-conservative amino acid substitutions, deletions, or additions. Those peptides containing one or more of naturally occurring amino acid derivatives of the 20 standard amino acids are also included as “derivatives”.

SEQ ID NO:1 consists of the sequence: MNLQPIFWIGLISSVCCVFA corresponding to amino acids 1 to 20 of human integrin β1 as defined in the Uniprot database entry with accession number P05556-1, version 241 dated 31 Jul. 2019.

As it is used herein in relation to the polypeptide comprising sequence SEQ ID NO:1, the term “functionally equivalent variant” refers to a corresponding amino acid sequence containing at least one difference in amino acids (substitution, insertion, or elimination) in comparison with the reference sequence. In certain embodiments, a “variant” has a high amino acid sequence homology and/or conservative amino acid substitutions, eliminations, and/or insertions in comparison with the reference sequence. In some embodiments, a variant has no more than 15, 12, 10, 9, 8, 7, 6, 5, 4, 3, 2, 1 differences in amino acids in comparison with the reference sequence.

Suitable functional variants are those showing a degree of amino acid sequence identity with respect to the peptide of SEQ ID NO: 1 of at least 25%, such as 25%, 30%, 40%, 50%, 60%, 65%, 70%, 75%, 80%, 85%, 90%, 91%, 92%, 93%, 94%, 95%, 96%, 97%, 98%, 99%. The degree of identity between two polypeptides is determined using computer algorithms and methods widely known by those skilled in the art. The identity between two amino acid sequences is preferably determined by means of using the BLASTP algorithm as described previously (BLAST manual, Altschul, S., et al., NCBI NLM NIH Bethesda, Md. 20894, Altschul, S., et al., J. Mol. Biol. 1990; 215: 403-410). In a preferred embodiment, sequence identity is determined through the full length of the polypeptide of SEQ ID NO: 1, or through the full length of the variant, or of both. Functionally equivalent variants of the polypeptide of the invention may also include post-translational modifications, such as glycosylation, acetylation, isoprenylation, myristoylation, proteolytic processing, etc.

Alternatively, suitable functional variants of the present invention are those in which one or more positions within the polypeptide of the invention contain an amino acid which is a conservative or non-conservative substitution of the amino acid present in sequence SEQ ID NO: 1.

The term “amino acid” or “residue” refers to naturally occurring and synthetic amino acids, as well as to amino acid analogs and amino acid mimetics that work in a manner similar to naturally occurring amino acids. Amino acids can be referred to herein either by means of their commonly known three-letter symbols or by means of the one-letter symbols recommended by the IUPAC-IUB Commission on Biochemical Nomenclature.

The term amino acid includes naturally occurring amino acids (Ala, Arg, Asn, Asp, Gln, Glu, Gly, His, Ile, Leu, Lys, Met, Phe, Pro, Ser, Thr, Trp, Tyr, Val), uncommon natural amino acids, and non-natural (synthetic) amino acids. The amino acids are preferably in the L configuration, but the D configuration or mixtures of amino acids in D and L configurations are also considered.

The term “natural amino acids” comprises aliphatic amino acids (glycine, alanine, valine, leucine, and isoleucine), hydroxylated amino acids (serine and threonine), sulfated amino acids (methionine), dicarboxylic amino acids and their amides (aspartic acid, asparagine, glutamic acid, and glutamine), amino acids having two basic groups (lysine, arginine, and histidine), aromatic amino acids (phenylalanine, tyrosine, and tryptophan), and cyclic amino acids (proline).

As it is used herein, the term “non-natural amino acid” refers to a carboxylic acid or a derivative thereof, substituted with an amine group and structurally related to a natural amino acid. Non-limiting illustrative examples of modified or uncommon amino acids include 2-aminoadipic acid, 3-aminoadipic acid, beta-alanine, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, N-methylisoleucine, 6-N-methyl-lysine, N-methylvaline, norvaline, norleucine, ornithine, etc.

Non-limiting illustrative examples of uncommon amino acids are hydroxylysine and hydroxyproline, thyroxine, N-methylarginine, and N-acetyl-lysine.

“Conservative amino acid substitutions” result from the replacement of one amino acid with another one having similar structural and/or chemical properties such that the peptide or polypeptide resulting from said replacement conserves the same charge, structure, polarity, hydrophobicity/hydrophilicity and/or conserves functions such as amyloid peptide recognition, binding, and/or reduction capacity. For example, the following six groups each contains amino acids which are conservative substitutions of one another: 1) alanine (A), serine (S), threonine (T); 2) aspartic acid (D), glutamic acid (E); 3) asparagine (N), glutamine (Q); 4) arginine (R), lysine (K); 5) isoleucine (I), leucine (L), methionine (M), valine (V); and 6) phenylalanine (F), tyrosine (Y), tryptophan (W). The selection of such conservative amino acid substitutions is within the capability of one having ordinary skill in the art and is described, for example, by Dordo et al. (J. Mol. Biol, 1999, 217; 721-739) and Tailor et al. (J. Theor. Biol., 1986, 119:205-218). Such conservative amino acid modifications are based on the relative similarity of side chain amino acid substituents, for example, their hydrophobicity, hydrophilicity, charge, size, and the like. Exemplary conservative substitutions which take into consideration several of the preceding characteristics are well known to one skilled in the art.

The variants of the polypeptide of the present invention may also include non-conservative substitutions or non-conserved amino acid residues, i.e., the substitution of amino acids of sequence SEQ ID NO: 1 with another natural or non-natural amino acid having different electrochemical or steric properties. Therefore, the side chain of the amino acid substituent can be larger or smaller than the side chain of the native amino acid and with different functional groups. Examples of non-conservative substitutions include the substitution of phenylalanine or cyclohexylmethylglycine with alanine, isoleucine with glycine, or —NH—CH[(—CH₂)₅—COOH]—CO— with aspartic acid. Non-conservative substitutions included within the scope of the present invention are those which maintain the protective capacity of the polypeptide of the invention against amyloidosis. Assays for testing said protective capacity have been mentioned above in relation to the identification of functionally equivalent variants.

The variants of the invention may cover analog peptides, derivatives, salts, retro-inverso isomers, mimics, mimetics, or peptidomimetics (typically, synthetically synthesized peptides), as well as peptoids and semipeptoids.

“Analogs,” “derivatives,” and “mimetics” include molecules mimicking the chemical structure of a peptide structure and maintain the functional properties of the peptide structure. Approaches for designing peptide analogs, derivatives, and mimetics are known in the art. See, for example, Farmer, P. S. in Dmg Design (E. J. Ariens, ed.) Academic Press, New York, 1980, vol. 10, pp. 119-143; Ball, J. B. and Alewood, P. F. (1990) J. Mol. Recognition 3:55. Morgan, B. A. and Gainor, J. A. (1989) Ann. Rep. Med. Chem. 24:243; and Freidinger, R. M. (1989) Trends Pharmacol. Sci. 10:270. See also Sawyer, T. K. (1995) Peptidomimetic Design and Chemical Approaches to Peptide Metabolism in Taylor, M. D. and Amidon, G. L. (eds.) Peptide-Based Drug Design: Controlling Transport and Metabolism, Chapter 17; Smith, A. B. 3rd, et al. (1995) J. Am. Chem. Soc. 117: 11113-11123; Smith, A. B. 3rd, et al. (1994) J. Am. Chem. Soc. 116:9947-9962; and Hirschman, R., et al. (1993) J. Am. Chem. Soc. 115: 12550-12568.

A “derivative” (for example, a peptide or amino acid) includes forms in which one or more reactive groups in the compound have been derivatized with a substituent group. Examples of peptide derivatives include peptides in which an amino acid side chain, the peptide backbone, or the amino or carboxyl end has been derivatized (for example, peptide compounds with methylated amide bonds). An “analog” of compound X includes compounds which maintain the chemical structures required for functional activity but also contain certain different chemical structures. One example of a natural peptide analog is a peptide including one or more non-natural amino acids.

A “mimetic” of a compound includes compounds in which the chemical structures of the compound required for functional activity have been replaced by other chemical structures mimicking the compound conformation. Examples of peptidomimetics include peptide compounds in which the peptide backbone is substituted with one or more benzodiazepine molecules (see, for example, James, G L et al. (1993) Science 260: 1937-1942) or oligomers mimicking peptide secondary structure by means of the use of amide bond isosteres and/or modification of the native peptide primary chain, including chain elongation or heteroatom incorporation; examples of such peptidomimetics include azapeptides, oligocarbamates, oligoureas, beta-peptides, gamma-peptides, oligo(phenylene ethynylene)s, vinyl sulfone peptides, poly-N-substituted glycines (peptoids), and the like. Methods for preparing peptidomimetic compounds are well known in the art and specified, for example, in Quantitative Drug Design, C. A. Ramsden Gd., Chapter 17.2, F. Choplin Pergamon Press (1992).

In addition to the foregoing, variants of the polypeptide of the present invention can also include one or more modified amino acids or one or more non-amino acid monomers (for example, fatty acids, complex carbohydrates, etc.) in relation to sequence SEQ ID NO: 1.

In a preferred embodiment, the peptide for use in the present invention comprises or consists of a truncated form of human integrin β1, wherein at least the amino acids corresponding to the sequence of SEQ ID NO:1, corresponding to amino acids 1 to 20 of human integrin ß1, as defined in the Uniprot database entry with accession number P05556-1, version 241 dated 31 Jul. 2019, are present. In a preferred embodiment, the peptide for use in the present invention comprises sequence or consists of SEQ ID NO:2, corresponding to amino acids 1 to 140 of human integrin ß1 as defined in the Uniprot database entry with accession number P05556-1, version 241 dated 31 Jul. 2019. In another preferred embodiment, the peptide for use in the present invention comprises or consists of sequence SEQ ID NO:3, corresponding to amino acids 1 to 371 of human integrin ß1 as defined in the Uniprot database entry with accession number P05556-1, version 241 dated 31 Jul. 2019. In another preferred embodiment, the peptide for use in the present invention comprises or consists of sequence SEQ ID NO:4, corresponding to amino acids 1 to 728 of human integrin ß1 as defined in the Uniprot database entry with accession number P05556-1, version 241 dated 31 Jul. 2019. In another preferred embodiment, the peptide according to the present invention does not comprise the complete sequence of a ß1 integrin. In another embodiment, the peptide according to the present invention does not comprise the complete sequence of a human ß1 integrin. In another embodiment, the peptide according to the present invention does not comprise the complete sequence of a human integrin ß1 corresponding to the 798 amino acids of human integrin ß1 as defined in the Uniprot database entry with accession number P05556-1, version 241 dated 31 Jul. 2019.

In some embodiments, the polypeptide according to the present invention has a length of 768 amino acids or less. In some embodiments, the polypeptide according to the present invention has a length of about 750 amino acids or less, of about 700 amino acids or less, of about 650 amino acids or less, of about 600 amino acids or less, of about 550 amino acids or less, of about 450 amino acids or less, of about 400 amino acids or less, of about 350 amino acids or less, of about 300 amino acids or less, of about 250 amino acids or less, of about 200 amino acids or less, of about 150 amino acids or less, of about 100 amino acids or less, of about 90 amino acids or less, of about 80 amino acids or less, of about 70 amino acids or less, of about 60 amino acids or less, of about 50 amino acids or less, of about 40 amino acids or less, of about 30 amino acids or less or of about 25 amino acids or less

One skilled in the art will understand that the polypeptide according to the present invention may contain, in addition to at least one human integrin ß1 region comprising amino acids 1 to 20, additional sequences that are not derived from the human integrin ß1 sequence which may facilitate putting the method of the invention into practice.

For example, the polypeptide and variants of the present invention may also comprise non-amino acid moieties, such as, for example, peptide-linked hydrophobic moieties (various linear, branched, cyclic, polycyclic, or heterocyclic hydrocarbons and hydrocarbon derivatives); several protective groups, particularly where the compound is linear, linked to the terminals of the compound to reduce degradation. Suitable functional protective groups are described in Green and Wuts, “Protective groups in organic synthesis,” John Wiley and Sons, Chapters 5 and 7, 1991.

The (non-amino acid) chemical groups present in the variants can be included to improve various physiological properties such as: to reduce degradation or clearance; to reduce repulsion by various cellular pumps, to improve immunogenic activities, to improve various modes of administration (such as the binding of various sequences which allow penetration through various barriers, through the intestine, etc.); increased specificity, increased affinity, increased stability, bioavailability, solubility, reduced toxicity, and the like.

In a particular embodiment, in order to facilitate the uptake of the peptide or polypeptide for use according to the present invention, the peptide or polypeptide can be modified by means of fusion thereof with compounds facilitating its uptake through cell membranes or facilitating its translocation through the blood-brain barrier (BBB).

In one embodiment, the chemical moiety facilitating the cellular uptake of the polypeptide is a lipid or a fatty acid.

A fatty acid is generally a molecule comprising a carbon chain with an acid moiety (for example, carboxylic acid) at one end of the chain. The carbon chain of a fatty acid may be of any length, however, the carbon chain is preferably at least 2, 3, 4, 5, 6, 7, 8, 9, 10, 11, 12, 13, 14, 15, 16, 17, 18, 19, 20, or more carbon atoms in length, and any interval that can be derived therefrom. In specific embodiments, the carbon chain is from 4 to 18 carbon atoms in length in the part of the fatty acid chain. In specific embodiments, the fatty acid carbon chain comprises an odd number of carbon atoms, however, an even number of carbon atoms in the chain may be preferred in specific embodiments. A fatty acid comprising only single bonds in its carbon chain is referred to as saturated, whereas a fatty acid comprising at least one double bond in its chain is referred to as unsaturated. The fatty acid can be branched, although in preferable embodiments of the present invention, it is not branched. The specific fatty acids include, but are not limited to, linoleic acid, oleic acid, palmitic acid, linolenic acid, stearic acid, lauric acid, myristic acid, arachidic acid, palmitoleic acid, arachidonic acid.

In a preferred embodiment, the chemical moiety facilitating the cellular uptake of the polypeptide is a cell-penetrating peptide sequence, in which case, the conjugate is a fusion protein comprising a polypeptide of the invention or the functionally equivalent variant of said polypeptide and the cell-penetrating peptide sequence.

The term “fusion protein” refers to proteins generated by means of gene technology consisting of two or more functional domains derived from different proteins. A fusion protein can be obtained by conventional means, for example, by the gene expression of the nucleotide sequence encoding said fusion protein in a suitable cell. It will be understood that the cell-penetrating peptide refers to a cell-penetrating peptide which is different from the cell-penetrating peptide that is part of the polypeptide of SEQ ID NO: 1 or of the functionally equivalent variant of said polypeptide.

The term “cell-penetrating peptide sequence” is used interchangeably herein with “CPP,” “protein transduction domain,” or “PTD”. It refers to a peptide chain of variable length which directs protein transport within a cell. It is usually taken into the cell by means of endocytosis, but the peptide can also be internalized into the cell by means of direct membrane translocation. CPPs usually have an amino acid composition either containing a high relative abundance of positively charged amino acids such as lysine or arginine or having sequences containing an alternating pattern of polar/charged amino acids and non-polar, hydrophobic amino acids. Examples of CPP which can be used in the present invention include, without limitation, the CPP found in the protein of Drosophila antennapedia (RQIKIWFQNRRMKWKK; SEQ ID NO: 5), the CPP found in herpes simplex virus 1 (VHS-1) VP22 DNA-binding protein (DAATATRGRSAASRPTERPRAPARSASRPRRPVE; SEQ ID NO: 6), the CPP of Bac-7 (RRIRPRPPRLPRPRPRPLPFPRPG; SEQ ID NO: 7), the CPPs of HIV-1 TAT protein consisting of amino acids 49-57 (RKKRRQRRR; SEQ ID NO: 8), amino acids 48-60 (GRKKRRQRRRTPQ; SEQ ID NO: 9), amino acids 47-57 (YGRKKRRQRRR; SEQ ID NO: 10), the CPP of 5413-PV peptide (ALWKTLLKKVLKAPKKKRKV; SEQ ID NO: 11), the CPP of penetratin (RQIKWFQNRRMKWKK; SEQ ID NO: 12), the CPP of SynB1 (RGGRLSYSRRRFSTSTGR; SEQ ID NO: 13), the CPP of SynB3 (RRLSYSRRRF; SEQ ID NO: 14), the CPP of PTD-4 (PIRRRKKLRRLK; SEQ ID NO: 15), the CPP of PTD-5 (RRQRRTSKLMKR; SEQ ID NO: 16), the CPP of FHV coat-(35-49) (RRRRNRTRRNRRRVR; SEQ ID NO: 17), the CPP of BMV Gag-(7-25) (KMTRAQRRAAARRNRVVTAR; SEQ ID NO: 18), the CPP of HTLV-II Rex-(4-16) (TRRQRTRRARRNR; SEQ ID NO: 19), the CPP of D-Tat (GRKKRRQRRRPPQ; SEQ ID NO: 20), the CPP of R9-Tat (GRRRRRRRRRPPQ; SEQ ID NO: 21), the CPP of MAP (KLALKLALKLALALKLA; SEQ ID NO: 22), the CPP of SBP (MGLGLHLLVLAAALQGAWSQPKKKRKV; SEQ ID NO: 23), the CPP of FBP (GALFLGWLGAAGSTMGAWSQPKKKRKV; SEQ ID NO: 24), the CPP of MPG (ac-GALFLGFLGAAGSTMGAWSQPKKKRKV-cya; SEQ ID NO: 25), the CPP of MPG(ENLS) (ac-GALFLGFLGAAGSTMGAWSQPKSKRKV-cya; SEQ ID NO: 26), the CPP of Pep-1 (ac-KETVWVETVWVTEWSQPKKKRKV-cya; SEQ ID NO: 27), the CPP of Pep-2 (ac-KETWFETWFTEWSQPKKKRKV-cya; SEQ ID NO: 28), a polyarginine sequence having the R_{N} structure (in which N is between 4 and 17), the sequence GRKKRRQRRR (SEQ ID NO: 29), the sequence RRRRRRLR (SEQ ID NO: 30), the sequence RRQRRTSKLMKR (SEQ ID NO: 31); transportan GWTLNSAGILLGKINLKALAALAKKIL (SEQ ID NO: 32); KALAWEAKLAKALAKALAKHLAKALAKALKCEA (SEQ ID NO: 33); RQIKIWFQNRRMKWKK (SEQ ID NO: 34), the sequence YGRKKRRQRRR (SEQ ID NO: 35); the sequence RKKRRQRR (SEQ ID NO: 36); the sequence YARAAARQARA (SEQ ID NO: 37); the sequence THRLPRRRRRR (SEQ ID NO: 38); the sequence GGRRARRRRRR (SEQ ID NO: 39).

In some embodiments, the fusion protein of the invention may comprise an additional chemical moiety including, among others, fluorescent groups, biotin, polyethylene glycol (PEG), amino acid analogs, non-natural amino acids, phosphate groups, glycosyl groups, radioisotope markers, and pharmaceutical molecules. In other embodiments, the polypeptide may comprise one or more chemically reactive groups including, among others, ketone, aldehyde, Cys residues, and Lys residues.

In a particular embodiment, the fusion proteins of the invention comprise a tag attached to the C-terminal or N-terminal domain of said fusion protein or variant of said polypeptide. Said tag is generally a peptide or amino acid sequence which can be used in the isolation or purification of said fusion protein. Therefore, said tag can be attached to one or more ligands, for example, one or more ligands of an affinity matrix such as a high affinity chromatography bead or support. An example of said tag is a histidine tag (His tag or HT), such as a tag comprising 6 histidine residues (His6 or H6), which can be attached to a high affinity nickel (Ni²⁺) or cobalt (Co²⁺) column. The His tag has the desirable characteristic of being able to be attached to its ligands under conditions which are denaturing for most proteins and destabilizing for most protein-protein interactions. Therefore, it can be used to remove the H6-labeled bait protein after destabilization of the protein-protein interactions in which the bait participated.

Additional non-limiting illustrative examples of tags useful for isolating or purifying a fusion protein include an Arg tag, FLAG tag (DYKDDDDK; SEQ ID NO: 40), Strep tag (WSHPQFEK; SEQ ID NO: 41), an epitope that can be recognized by an antibody, such as the c-myc tag (recognized by an anti-c-myc antibody), HA tag (YPYDVPDYA; SEQ ID NO: 42), V5 tag (GKPIPNPLLGLDST; SEQ ID NO: 43), SBP tag, S tag, calmodulin-binding peptide, cellulose-binding domain, chitin-binding domain, glutathione-S-transferase tag, maltose-binding protein, NusA, TrxA, DsbA, Avi tag, etc. (Terpe K., Appl. Microbiol. Biotechnol. 2003, 60:523-525), an amino acid sequence such as AHGHRP (SEQ ID NO: 44) or PIHDHDHPHLVIHSGMTCXXC (SEQ ID NO: 45), β-galactosidase, and the like.

The tag can be used, if desired, for isolating or purifying said fusion protein.

The polypeptide or the variants according to the invention can be attached to a functional component. This functional component can be a molecule which causes the compound to be directed to specific organs, cells, or molecules, such as a hormone, an antibody, a transcription factor, or another protein molecule; or it can be a marker as described above, such as a group or atom containing a radioactive or magnetically active nucleus; or it could be a fluorescent group, a colored group, or another group detectable by spectroscopy; or it could be a group which contains an unpaired electron and acts as a spin marker, such as the 2,2,5,5-tetramethyl-1-pyrrolidinyloxy (PROXYL) group or the 2,2,6,6-tetramethyl-1-piperidinyloxy (TEMPO) group.

The polypeptide or the variants of the invention are attached to any of these functional components or to any other functional component by means of an amide bond, an ester bond, or any other suitable bond between a side chain, an N substituent, or any of the two ends of the complete peptide. The functional component and this bond are generated before, during, or after the synthesis of the complete peptide by means of the coupling of the suitable molecules. For example, the inclusion of a cysteine or lysine moiety in the complete peptide allows it to be attached to a functional component containing an electrophilic group such as a bromine or iodine group, or an ester or anhydride group, by means of the sulfur atom of the thiol of the cysteine moiety or the nitrogen atom of the amino of the lysine moiety mounting a nucleophilic attack against that electrophilic group of the functional component. Alternatively, a bifunctional cross-linking agent can be used to attach the peptide to the functional component; or the complete peptide can be synthesized using a specially prepared amino acid derivative that already contains the functional component; or a standard coupling agent such as dicyclohexylcarbodiimide can be used to form an amide bond between a side chain or a terminal carboxyl or amino group of the peptide and a carboxyl or amino group of the functional component.

The activity or function of the polypeptides of the invention comprising sequence SEQ ID NO: 1 and the functionally equivalent variants thereof can be determined by means of any of the assays of the examples of the present application, in which the capacity of said peptide to avoid cell death is measured. Non-limiting examples include the cell culture assay of the inhibition of β amyloid peptide 1-42-mediated Rac GTPase activation, reduction of β amyloid peptide 1-42-induced reactive oxygen species (ROS) generation, prevention and/or reduction of β amyloid peptide 1-42-induced reactive astrogliosis, or reduction of GRP78 protein expression, among others. One skilled in the art will be able to design additional experiments that can evaluate the protective capacity of the peptide of the invention or the functionally equivalent variants thereof against amyloid peptide-induced cell death.

The term “treat” or “treatment” means both therapeutic and prophylactic treatment or preventive measures, in which the purpose is to prevent or slow down (reduce) an undesired physiological change or disorder, such as cell death associated with the formation of amyloid deposits and/or more specifically with Aβ peptide (1-42)-induced neuronal death in the central nervous system (CNS). For the purpose of this invention, beneficial or desired clinical results include, without limitation, relief of symptoms, reduction of the extent of the disease, stabilized pathological state (specifically, the pathological state is not worsened), delay or slowing down of disease progression, improvement or palliation of the pathological state and (both partial and complete) remission, both detectable and undetectable. “Treatment” can also mean a prolonged survival in comparison with the expected survival if no treatment is received. Those subjects in need of treatment include those subjects already suffering from the condition or disorder, as well as those susceptible to suffering from the condition or disorder, or those in which the condition or disorder must be prevented.

Treatment includes administering to an individual in need of said treatment a polypeptide comprising sequence SEQ ID NO:1 or a functionally equivalent variant thereof according to the invention.

The term “amyloid deposit” refers to the extracellular deposition (accumulation) of deposits formed by amyloid protein aggregation followed by the subsequent combination of aggregates and/or amyloid proteins, formation of amyloid deposits, and the like.

As they are used herein, the terms “amyloid protein,” “amyloid,” “amyloid fibrils,” and “amyloid fibers” are generic terms for a tertiary structure formed by the aggregation of any of several different proteins and consisting of an ordered arrangement of sheets stacked perpendicular to a fiber axis. An example of amyloid is the β amyloid aggregate formed in Alzheimer's disease, which is made up of the beta-amyloid “βA” peptide, which are internal fragments of 39-43 amino acids cleaved from human amyloid precursor protein (hAPP). Other exemplary amyloid proteins include β-synuclein with folding abnormality (associated with Parkinson's disease), huntingtin (associated with Huntington's disease), tau (associated with Alzheimer's disease), and abnormal PrPSc prion protein conformation. Additional examples are known to those skilled in the art (see, for example, Aguzzi (2010), and Eichner and Radford, Mol. Cell (2011) 43: 8-18). Therefore, unless a protein or a peptide is specified, the use of the terms “amyloid,” “amyloid fibrils,” or “amyloid fibers” must not be interpreted as being limited to any particular protein or disease.

The term “diseases associated with the formation of amyloid deposits,” or “diseases associated with amyloidosis,” or “amyloid diseases” includes, but is not limited to, diseases associated with systemic, local, chronic, and senile amyloidosis. Amyloidoses are characterized by “amyloid deposits” which are made up of amyloid fibrils as previously described. Alzheimer's disease (AD) is one of the most well-known examples of amyloidosis. Amyloid fibrils have in common a series of characteristics although they are formed by different proteins;, they all have an elongated morphology, are stained with Congo Red, and exhibit a characteristic X-ray diffraction pattern referred to as “cross-beta”. The fibrils are formed by the cooperative binding of molecules with a characteristic formation kinetics including a slower initial nucleation phase and a quicker subsequent elongation or growth phase.

There are different types of amyloid deposits including, among others:
amyloid fibril deposits which are associated with dementia in AD, with dementia associated with Lewy bodies, with Down syndrome, with Guam dementia complex associated with parkinsonism, with hereditary cerebral hemorrhage with amyloidosis-Dutch type, and with other similar processes (in which the specific amyloid refers to the amyloid precursor protein or APP);
amyloidosis associated with chronic inflammation, for example, osteomyelitis, tuberculosis, familial Mediterranean fever, hereditary cerebral hemorrhage, rheumatoid arthritis, Crohn's disease, ankylosing spondylitis, Castleman disease, and the like (in which the specific amyloid refers to AA type amyloid or amyloid A protein (AA protein), with a structure that is different from an immunoglobulin and made up of 76 amino acids with a molecular weight of 8500 Daltons; the AA protein is derived from an HDL3 lipoprotein-bound, circulating liver synthesis precursor referred to as “serum amyloid A protein precursor” (SAP));
amyloidosis associated with multiple myeloma, for example, with B-cell dyscrasias and the like (in which the specific amyloid refers to AL type amyloid formed by immunoglobulin light chains with Lambda (λ) predominating over Kappa (κ) in a proportion of 2:1, with a tendency to form fibrillar structures adopting a folded beta distribution);
amyloidosis associated with type 2 diabetes (in which the specific amyloid is pancreatic islet amylin);
amyloidosis associated with prion diseases, for example, Creutzfeldt-Jacob disease, kuru, Gerstmann-Sträussler-Scheinker disease, animal scrapie, and the like (in which the specific amyloid refers to PrP or prion protein amyloid);
amyloidosis associated with chronic hemodialysis, amyloidosis associated with long-term hemodialysis, with carpal tunnel syndrome, and with other similar processes (in which the specific amyloid refers to ss2-microglobulin);
senile cardiac amyloidosis, familial amyloid polyneuropathy, and similar processes (in which the specific amyloid refers to transthyretin or prealbumin); and
amyloidosis associated with endocrine tumors such as medullary thyroid carcinoma and with other similar processes (in which the specific amyloid is a procalcitonin variant).

In a particular embodiment, the disease associated with amyloid deposit includes, but is not limited to, Alzheimer's disease, dementia associated with Lewy bodies, Down syndrome, Guam dementia complex associated with parkinsonism, hereditary cerebral hemorrhage with amyloidosis-Dutch type, amyloidosis associated with chronic inflammation, for example, osteomyelitis, tuberculosis, familial Mediterranean fever, hereditary cerebral hemorrhage, rheumatoid arthritis, Crohn's disease, ankylosing spondylitis, Castleman disease, amyloidosis associated with multiple myeloma, for example, B-cell dyscrasias and the like, amyloidosis associated with type 2 diabetes, amyloidosis associated with prion diseases, for example, Creutzfeldt-Jacob disease, kuru, Gerstmann-Sträussler-Scheinker disease, animal scrapie, and the like, amyloidosis associated with chronic hemodialysis, amyloidosis associated with long-term hemodialysis, carpal tunnel syndrome, senile cardiac amyloidosis, familial amyloid polyneuropathy, or amyloidosis associated with endocrine tumors such as medullary thyroid carcinoma.

In a particular embodiment, the amyloid deposits are extracellular deposits of beta amyloid peptide or Aβ peptide. In another more particular embodiment, the amyloid deposits are Aβ peptide (1-42) deposits.

“Beta amyloid peptide” or “Aβ peptide” is understood to mean a peptide of 39 to 43 amino acids derived from APP (NM-000484.3 variant 1, NM_201413.2 variant 2, NM_201414.2 variant 3, NM_001136129.2 variant 5, NM_001136130.2 variant 6). In physiological conditions, this peptide is generated from APP by means of APP processing through the amyloidogenic pathway.

“Amyloid precursor protein” or “APP” is a transmembrane glycoprotein which is a substrate of the family of proteases referred to as presenilins. Depending on the proteases participating in APP processing, reaction products with a different activity will be generated at the level of neuronal physiology.

“Beta amyloid peptide (1-42)” or “Aβ peptide (1-42)” refers to an Aβ peptide made up of the first 42 amino acids resulting from APP processing through the amyloidogenic pathway (Zain S b et al., Proc. Natl. Acad. Sci. USA. 1988 February; 85(3):929-933).

In a particular embodiment, the diseases associated with Aβ peptide amyloid deposits include, but are not limited to, AD, memory loss, attention deficit symptoms associated with AD, diffuse Lewy body-type AD, mild cognitive impairment, hereditary cerebral hemorrhage with amyloidosis-Dutch type, β-amyloid angiopathy, and cerebral hemorrhage such as cerebral hemorrhage due to solitary cerebral amyloid angiopathy, prion infections, type II diabetes, degenerative dementias, including dementias of mixed degenerative and vascular origin, frontotemporal dementia, presenile dementia, senile dementia, dementia associated with AIDS, parkinsonian disorders such as Parkinson's disease (PD), subacute sclerosing panencephalitis parkinsonism, post-encephalitic parkinsonism, pugilistic encephalitis, Guam parkinsonism-dementia complex, Pick's disease, multiple system atrophy (MSA), progressive supranuclear paralysis (PSP) and corticobasal degeneration (CBD), Down syndrome, Lewy body disease, Huntington's disease, amyotrophic lateral sclerosis, multiple sclerosis, and neurotraumatic diseases such as acute stroke, epilepsy, mood disorders such as depression, schizophrenia and bipolar disorders, ischemia, brain injury, particularly traumatic brain injury, inflammation, and chronic inflammatory diseases.

“Amyloid peptide or Aβ peptide-induced cell death” is understood to mean the response, in the form of cell death, of cells which have been exposed to the amyloid peptide or Aβ peptide. In a particular embodiment, the Aβ peptide-induced cell death is through the PI3K/PKD1/new PKC/Rac1/cell death pathway. In a preferred embodiment, the cell death is neuronal death and is induced by the Aβ peptide through the PI3K/PKD1/new PKC/Rac1/neuronal death pathway. In another particular embodiment, the cell death or neuronal death is caused by the Aβ peptide (1-42) through the PI3K/PKD1/new PKC/Rac1/cell death or neuronal death pathway.

One skilled in the art will understand that the polypeptide of the invention for use in the treatment of a disease associated with the formation of amyloid deposits will be capable of reducing amyloid load. Likewise, one skilled in the art understands that said peptide can be neuroprotective or exhibit neuroprotective properties.

In one embodiment, the polypeptides comprising amino acids 1-20 of integrin β1 (SEQ ID NO: 1) or a functionally equivalent variant are capable of reducing amyloid load. As it is used herein, a protein which “reduces amyloid load” is capable of carrying out one or more of the following functions: it inhibits amyloid formation, causes amyloid disaggregation, promotes amyloid clearance, inhibits amyloid aggregation, blocks and/or prevents toxic amyloid oligomer formation, and/or promotes toxic amyloid oligomer elimination. In a particular embodiment, the peptide capable of reducing amyloid load comprises sequence SEQ ID NO: 2, SEQ ID NO: 3, or SEQ ID NO: 4.

As it is used herein, the term “neuroprotective” refers to the attenuation of the effects of neuronal degeneration or death by means of any mechanism known or yet to be known, for example, necrosis, apoptosis, autophagy, oxidative damage, by-product deposition, cell architecture loss, etc., or to the disappearance of the effects of neuronal degeneration or death by means of any mechanism known or yet to be known, for example, necrosis, apoptosis, autophagy, oxidative damage, by-product deposition, cell architecture loss, etc., or to the decrease or disappearance of the side effects thereof.

In a particular embodiment, the neuroprotective polypeptide comprises sequence SEQ ID NO: 1 or a functionally equivalent variant. In another embodiment, the neuroprotective polypeptide comprises sequence SEQ ID NO: 2, SEQ ID NO: 3, or SEQ ID NO: 4.

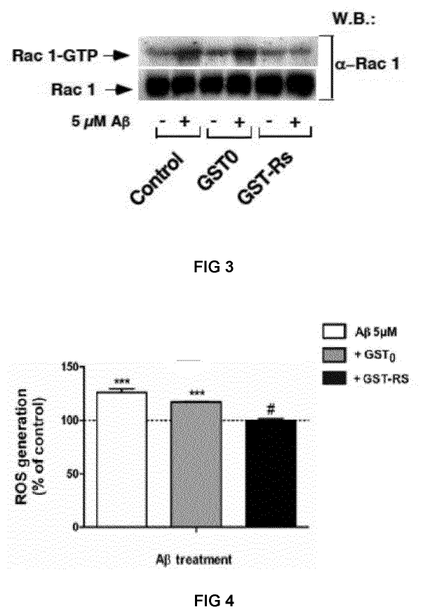

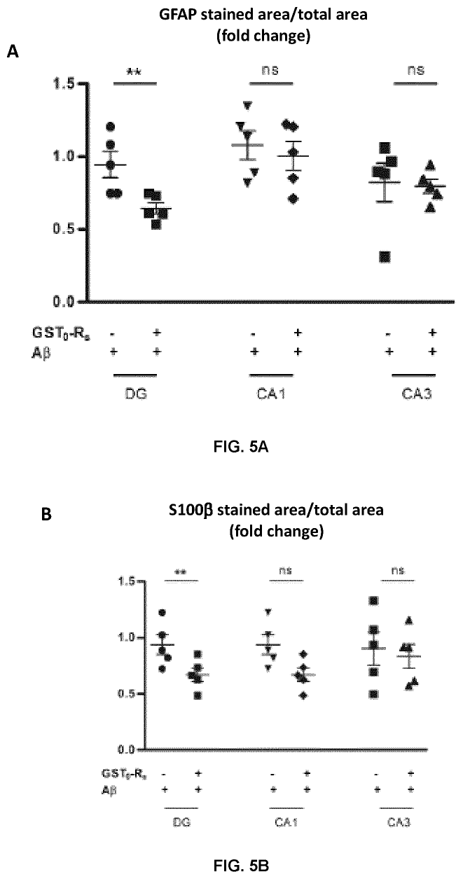

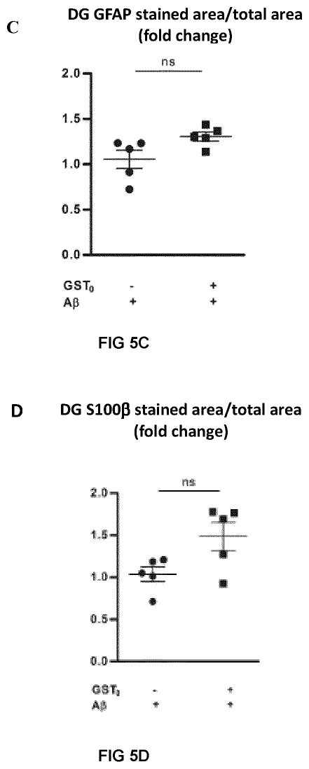

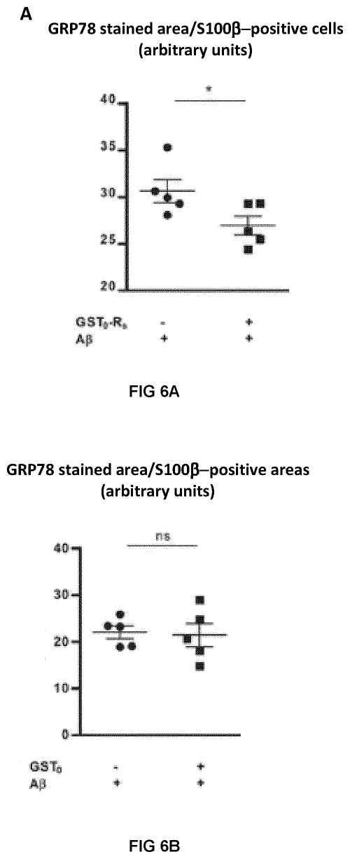

In a particular embodiment, the polypeptide of the invention comprising sequence SEQ ID NO: 1 is capable of inhibiting β amyloid peptide 1-42-mediated Rac GTPase activation, as shown in FIG. 3. In another particular embodiment, the polypeptide comprising sequence SEQ ID NO: 1 is capable of deducing β amyloid peptide 1-42-induced reactive oxygen species (ROS) generation (FIG. 4). In another particular embodiment, the polypeptide comprising sequence SEQ ID NO: 1 prevents and/or reduces β amyloid peptide 1-42-induced reactive astrogliosis (FIG. 5). In another embodiment, the polypeptide comprising sequence SEQ ID NO: 1 is capable of reducing β amyloid peptide 1-42-induced GRP78 protein expression (FIG. 6).

In a particular embodiment, the polypeptide of the invention comprising sequence SEQ ID NO: 1 is a polypeptide comprising sequence SEQ ID No: 2, SEQ ID NO: 3, or SEQ ID NO: 4.

In a particular embodiment, the disease is Alzheimer's disease.

Alzheimer's disease (AD) is a neurological disorder considered as mainly being caused by amyloid plaques, an abnormal protein deposit accumulation in the brain. The amyloid type most often found in the brain of affected individuals is primarily made up of Aβ fibrils. Scientific evidence demonstrates that an increased beta-amyloid protein production and accumulation in plaques leads to neuronal cell death, which contributes to AD development and progression. Proteins primarily responsible for plaque formation include amyloid precursor protein (APP) and two presenilins (presenilin I and presenilin II). The sequential cleavage of amyloid precursor protein (APP), which is expressed constitutively and catabolized in most cells by enzymes 13 and secretase, leads to the release of an Aβ peptide of 39 to 43 amino acids. APP degradation probably increases the tendency thereof to aggregate, forming plaques. In principle, the Aβ fragment (1-42) has a high tendency to form aggregates due to two highly hydrophobic amino acid residues at its C-terminal end. Accordingly, it is considered that the Aβ fragment (1-42) is mainly involved in and responsible for the initiation of neuritic plaque formation in AD, and therefore has a high pathological potential. Alzheimer's disease is characterized by neuron and synapse loss in the cerebral cortex and certain subcortical regions. This loss results in gross atrophy of the affected regions, including the degeneration of the temporal lobe and parietal lobe, and parts of the frontal cortex and cingulate gyrus. Both amyloid plaques and neurofibrillary tangles are clearly observed by means of microscopy in the brains of people affected by AD. The plaques are mostly insoluble dense deposits of beta amyloid peptide and cellular material from the outside and the surroundings of the neurons. The tangles (neurofibrillary tangles) are aggregates of the microtubule-associated, hyperphosphorylated tau protein which has accumulated in cells.

Compositions of the Invention

In another aspect, the invention relates to a composition comprising a polypeptide comprising sequence SEQ ID NO: 1 or a functionally equivalent variant thereof and a compound suitable for the treatment of a disease associated with the formation of amyloid deposits.

The terms polypeptide, functionally equivalent variant, and disease associated with the formation of amyloid deposits have been described above in the context of the medical uses of the polypeptides of the invention.

Compounds suitable for the treatment of a disease associated with the formation of amyloid deposits are defined as those compounds which lead to, without limitation, relief of symptoms, reduction of the extent of the disease, stabilized pathological state (specifically, the pathological state is not worsened), delay or slowing down of disease progression, improvement or palliation of the pathological state and (both partial and complete) remission, both detectable and undetectable.

Non-limiting examples of compounds suitable for the treatment of a disease associated with the formation of amyloid deposits include, without limitation, Rac1, PKC, and PDK1 inhibitors.

In the present invention, “PKC” (or “protein kinase C”) or, in plural, PKCs, is understood to mean the members of a family of enzymes involved in controlling the function of other proteins through the phosphorylation of hydroxyl groups of serine and threonine amino acid residues in said proteins. PKCs are divided into three groups based on the structure and co-factor requirements (Jaken, S. 1996. Curr. Opin. Cell Biol. 8: 168): classic (or conventional) PKC isoforms (cPKC) which require diacylglycerol (DAG), Ca²⁺, and phosphatidylserine (PS) for optimal activity and include PKC-α (PRKCA), PKC-βI (PRKCB 1), PKC-βII, and PKC-γ (PRKCG); new PKC forms (nPKCs), including PKC-δ (PRKCD), PKC-ε (PRKCE), PKC-η (PRKCH), and PKC-Θ (PRKCQ) isoforms; these new PKCs require DAG and PS but not Ca²⁺; and atypical PKC forms, including PKC-ι (PRKCI), PKC-ζ (PRKCZ), PK-N 1 (PKN I), and PK-N2 (PKN2), bind to PS but are not sensitive to Ca²⁺ and DAG.

As it is used herein, the term “PKC” includes all PKC forms and isoforms.

In the context of the present invention, a “PKC inhibitor” is understood to mean a compound which reduces the activity of one (or several) PKC(s) in contact with said protein, as well as any substance or compound which is capable of preventing or blocking the transcription and/or translation of the PKC-encoding gene (i.e., preventing or blocking the expression of said gene), or which is capable of preventing the protein encoded by said gene from performing its function (activity); i.e., said term “PKC inhibitor” includes compounds acting at the RNA level (e.g., antisense oligonucleotides, shRNA, siRNA, etc.) or at the protein level (e.g., antibodies, peptides, small organic compounds, or small molecules, etc.).

By way of non-limiting illustration, PKC expression inhibitory agents suitable for use in the present invention include, among others, for example, PKC-encoding gene-specific antisense oligonucleotides, specific microRNAs, specific catalytic RNAs or ribozymes, specific interfering RNAs (siRNAs), RNAs with “decoy” activity, i.e., with the capacity to bind specifically to a (generally protein) factor which is important for the expression of the gene, such that the expression of the gene of interest, in this case PKC, is inhibited, etc. Likewise, non-limiting illustrative examples of PKC inhibitory agents capable of preventing a PKC protein from performing its function include PKC inhibitory peptides, antibodies targeted specifically against PKC epitopes essential for performing its function, such as the CI (DAG-binding) and C2 (Ca²⁺-binding) domains, ATP-binding regions, substrate-binding site, phosphorylation regions, etc. Likewise, various small chemical compounds (small molecules) which reduce the activity of PKC when they are contacted with said protein (small molecule inhibitors of PKC), for example, bisindolylmaleimide, chelerythrine, and the salts thereof, Floridzin, Rottlerin, GF109203X, Go 6976, CGP 41251, etc., which can be used in the present invention, are known. Additionally, other compounds capable of inhibiting PKC expression which can be used for putting the present invention into practice include aptamers and spiegelmers, i.e., single- or double-stranded D or L nucleic acids binding specifically to the target protein (in this case, PKC), resulting in a modification of the biological activity thereof. The aptamers and spiegelmers have a length of between 15 and 80 nucleotides, and preferably between 20 and 50 nucleotides.

PDK1 or PDPK1 (3-phosphoinositide dependent protein kinase 1) is a kinase which participates in various signaling pathways. The structure of PDK1 can be divided into two domains: the PH domain and the kinase or catalytic domain. The PH domain is mainly involved in the interaction of PDK1 with phosphatidylinositol (3,4)-bisphosphate and with phosphatidylinositol (3,4,5)-triphosphate, which is important for the localization and activation of some membrane-associated PDK1 substrates (e.g., AKT). The kinase domain has three ligand-binding sites: the substrate-binding site, the ATP-binding site, and the PIF pocket or coupling site. Many PDK1 substrates, including AKT and protein kinase C require binding to said coupling site [Frödin M, et al. 2002. EMBO J. 21 (20): 5396-407]. The human PDK1 gene is located in the 16pI3.3 region of chromosome 16 and has two transcription variants giving rise to two isoforms: isoform 1 (NM 002613.3, SEQ ID NO: 1) and isoform 2 (NM_031268.4, SEQ ID NO: 2).

In the context of the present invention, “PDK1 inhibitor” is understood to mean a compound which reduces the activity of PDK1 in contact with said protein, as well as any substance or compound which is capable of preventing or blocking the transcription and/or translation of the PDK1-encoding gene (i.e., preventing or blocking the expression of said gene), or which is capable of preventing the protein encoded by said gene from performing its function (activity); i.e., said term “PDK1 inhibitor” includes compounds acting at the RNA level (e.g., antisense oligonucleotides, shRNA, siRNA, etc.) or at the protein level (e.g., antibodies, peptides, small organic compounds, or small molecules, etc.).

By way of non-limiting illustration, PDK1 expression inhibitory agents suitable for use in the present invention include, among others, for example, PDK1-encoding gene-specific antisense oligonucleotides, specific microRNAs, specific catalytic RNAs or ribozymes, specific interfering RNAs (siRNAs), RNAs with “decoy” activity, i.e., with the capacity to bind specifically to a (generally protein) factor which is important for the expression of the gene, such that the expression of the gene of interest, in this case PDK1, is inhibited, etc. Likewise, non-limiting illustrative examples of PDK1 inhibitory agents capable of preventing the PDK1 protein from performing its function include PDK1 inhibitory peptides, antibodies targeted specifically against PDK1 epitopes essential for performing its function, or against PDK1, such as ATP-binding regions, the PIF pocket or coupling site, the substrate-binding site, or the PH domain, etc. Various small chemical compounds (small molecules) which reduce the activity of PDK1 when they are contacted with said protein [small molecule inhibitors of PDK1], for example, staurosporine and its derivatives, phenothiazine and its derivatives, urea derivatives, celecoxib and its derivatives, pyridinonylic compounds, polyheterocyclic compounds, etc., which can be used in the present invention, are known. Additionally, other compounds capable of inhibiting PDK1 expression which can be used for putting the present invention into practice include aptamers and spiegelmers, i.e., single- or double-stranded D or L nucleic acids binding specifically to the target protein (in this case, PDK1), resulting in a modification of the biological activity thereof. The aptamers and spiegelmers have a length of between 15 and 80 nucleotides, and preferably between 20 and 50 nucleotides.

In the context of the present invention, is “Rac1 inhibitor” understood to mean a compound which reduces the activity of Rac1 in contact with said protein, as well as any substance or compound which is capable of preventing or blocking the transcription and/or translation of the Rac1-encoding gene (i.e., preventing or blocking the expression of said gene), or which is capable of preventing the protein encoded by said gene from performing its function (activity); i.e., said term “PDK1 inhibitor” includes compounds acting at the RNA level (e.g., antisense oligonucleotides, shRNA, siRNA, etc.) or at the protein level (e.g., antibodies, peptides, small organic compounds, or small molecules, etc.).

By way of non-limiting illustration, RAC 1 expression inhibitory agents suitable for use in the present invention include, among others, for example, Rac1-encoding gene-specific antisense oligonucleotides, specific microRNAs, specific catalytic RNAs or ribozymes, specific interfering RNAs (siRNAs), RNAs with “decoy” activity, i.e., with the capacity to bind specifically to a (generally protein) factor which is important for the expression of the gene, such that the expression of the gene of interest, in this case Rac1, is inhibited, etc. Likewise, non-limiting illustrative examples of Rac1 inhibitory agents capable of preventing the Rac1 protein from performing its function include Rac1 inhibitory peptides, antibodies targeted specifically against Rac1 epitopes essential for performing its function, or against Rac1, such as the ATP-binding regions, the PIF pocket or coupling site, the substrate-binding site, or the PH domain, etc. Various small chemical compounds (small molecules) which reduce the activity of Rac1 when they are contacted with said protein (small molecule inhibitors of Rac1), for example, 6-mercaptopurine and its derivatives, GGTI-298, lovastatin, NSC23766, EHT 1864, and other compounds included in Table 1 (I), which can be used in the present invention, are known. Additionally, other compounds capable of inhibiting Rac1 expression which can be used for putting the present invention into practice include aptamers and spiegelmers, i.e., single- or double-stranded D or L nucleic acids binding specifically to the target protein (in this case, Rac1), resulting in a modification of the biological activity thereof. The aptamers and spiegelmers have a length of between 15 and 80 nucleotides, and preferably between 20 and 50 nucleotides.

Additionally, other compounds suitable for the treatment of a disease associated with the formation of amyloid deposits which can be included in the composition of the invention together with the polypeptide of the invention or functionally equivalent variants comprise, in a non-limiting manner, neutron transmission promoters, psychotherapeutic drugs, acetylcholinesterase inhibitors, calcium channel blockers, biogenic amines, benzodiazepine tranquilizers, synthesis promoters, acetylcholine conservation or release, acetylcholine postsynaptic receptor agonists, monoamine oxidase A or B inhibitors, N-methyl-D-aspartate glutamate receptor antagonists, non-steroidal anti-inflammatory drugs, antioxidants, and serotonergic receptor antagonists.

Likewise, the composition may comprise at least a different biologically active compound selected from the group consisting of anti-oxidative stress compounds, anti-apoptotic compounds, metal chelators, DNA repair inhibitors such as pirenzepine and metabolites, 3-amino-I-propanesulfonic acid (3APS), 1,3-propanedisulfonate (1,3PDS), secretase activators, β-secretase, and secretase inhibitors, tau proteins, neurotransmitters, anti-β sheet agents, anti-inflammatory molecules, or cholinesterase inhibitors (ChEI) such as tacrine, rivastigmine, donepezil, and/or galantamine, and other drugs and nutritional supplements, and optionally a pharmaceutically acceptable carrier, and/or diluent, and/or excipient.

Likewise, compounds suitable for the treatment of a disease associated with the formation of amyloid deposits may include niacin or memantine together with polypeptide according to the present invention, and optionally a pharmaceutically acceptable carrier, and/or diluent, and/or excipient.

Additionally, the composition comprises “atypical antipsychotics” such as, for example, clozapine, ziprasidone, risperidone, aripiprazole, or olanzapine for the treatment of positive and negative psychotic symptoms including hallucinations, delusions, thought disorders (manifested as significant incoherence, derailment, tangentiality), and strange and disorganized behavior, in addition to anhedonia, emotional blunting, apathy, and social isolation together with the polypeptide of the invention, and optionally a pharmaceutically acceptable carrier, and/or diluent, and/or excipient.

Other compounds which can be suitably used in the composition of the invention for the treatment of diseases associated with the formation of amyloid deposits are described, for example, in document WO2004/058258 (see particularly pages 16 and 17) including banks of therapeutic drugs (page 36-39), alkanesulfonic acids, and alkanesulfuric acid (pages 39-51), cholinesterase inhibitors (pages 51-56), NMDA receptor antagonists (pages 56-58), estrogens (pages 58-59), non-steroidal anti-inflammatory drugs (pages 60-61), antioxidants (pages 61-62), peroxisome proliferator-activated receptor (PPAR) agonists (pages 63-67), cholesterol reducing agents (pages 68-75); amyloid inhibitors (pages 75-77), amyloid formation inhibitors (pages 77-78), metal chelators (pages 78-79), antipsychotics and antidepressants (pages 80-82), nutritional supplements (pages 83-89), and compounds increasing the availability of the biologically active substances in the brain (see pages 89-93), and prodrugs (pages 93 and 94).

Additionally, the composition of the invention may include beta amyloid peptide-specific antibodies. Said antibodies can be monoclonal or polyclonal. Studies in transgenic animals have demonstrated that passive immunization with antibodies of this type, in addition to reducing the neuronal amyloidogenic load, improves cognitive deficits, even before the elimination of neuronal amyloid plaques. Non-limiting examples of antibodies of this type include, without limitation, bapineuzumab, solanezumab, gantenerumab, Crenezumab, PF-04360365 (Ponezumab), MABT5102A, GSK933776A, NI-101, SAR-228810, BAN-2401, AAB-003, aducanumab, Gamunex, MED11814, Octagam, or gammagard.

Other compounds suitable for the treatment of a disease associated with the formation of amyloid deposits are NMDA receptor antagonists. Non-limiting examples of NMDA receptor antagonists include, memantine, neramexane hydrochloride (MRZ 2/579; 1-amino-1,3,3,5,5-pentamethyl-cyclohexane hydrochloride), neramexane, or amantadine.

Additional compounds which may be present in the composition of the invention also include acetylcholinesterase inhibitors including, among others, galantamine, rivastigmine, donepezil, tacrine.

Likewise, amyloid peptide aggregation inhibitors such as synthetic glycosaminoglycan 3-amino-1-propanesulfonic acid (3APS, Alzhemed, tramiprosate), colostrinin, scyllo-inositol (ELND005), PBT1 (clioquinol), and PBT2, can be incorporated to the composition of the invention.

Additionally, compounds favoring amyloid aggregate and deposit elimination can be included in the composition of the invention. Said compounds would include, among others, activators of enzymes in charge of degrading amyloid plaques or compounds modulating the transport of beta amyloid peptide from the brain to peripheral circulation. Compounds modulating the transport of beta amyloid peptide from the brain to peripheral circulation include, among others, RAGE (receptor for advanced glycation end products) inhibitors such as PF-04494700 and TTP4000.

Other compounds suitable for the treatment of diseases associated with the formation of amyloid deposits which can be included in the composition of the invention comprise beta-secretase inhibitors (BACE1) for reducing the production of beta-amyloid peptide levels such as E2609, CTS21166, LY2886721, and MK-8931; gamma-secretase inhibitors and modulators such as semagacestat (LY450139), avagacestat, CHF5074, NIC5-15; alpha-secretase activators such as acitretin, epigallocatechin gallate, or bryostatin 1.

In a particular embodiment, the polypeptide comprising sequence SEQ ID NO: 1 present in the composition of the invention is a polypeptide comprising sequence SEQ ID NO: 2, SEQ ID NO: 3, or SEQ ID NO: 4.

In an additional embodiment, the compound suitable for the treatment of a disease associated with the formation of amyloid deposits is selected from the group consisting of:
(i) A cholinesterase inhibitor,
(ii) An NMDA receptor antagonist,
(iii) A beta amyloid peptide-specific antibody,
(iv) An β amyloid peptide aggregation inhibitor,

In particular embodiments:
(i) the cholinesterase inhibitor is selected from the group consisting of donepezil hydrochloride, rivastigmine, and galantamine,
(ii) the NMDA receptor antagonist is memantine,
(iii) the beta amyloid peptide-specific antibody is selected from the group consisting of solanezumab, bapineuzumab, and gantenerumab and/or
(iv) the β amyloid peptide aggregation inhibitor is selected from the group consisting of glycosaminoglycan 3-amino-1-propanesulfonic acid (3APS, tramiprosate), colostrinin, and scyllo-inositol.

In another aspect, the invention relates to the composition of the invention for use in medicine, and more particularly for use in the treatment of a disease associated with the formation of amyloid deposits.

In a particular embodiment, the disease associated with the formation of amyloid deposits is Alzheimer's disease, dementia associated with Lewy bodies, with Down syndrome, with Guam dementia complex associated with parkinsonism, with hereditary cerebral hemorrhage with amyloidosis-Dutch type, β-amyloid angiopathy, and cerebral hemorrhage such as cerebral hemorrhage due to solitary cerebral amyloid angiopathy osteomyelitis, tuberculosis, familial Mediterranean fever, hereditary cerebral hemorrhage, rheumatoid arthritis, Crohn's disease, ankylosing spondylitis, prion infections, Creutzfeldt-Jacob disease, type II diabetes, Castleman disease, amyloidosis associated with multiple myeloma, Parkinson's disease, subacute sclerosing panencephalitis parkinsonism, post-encephalitic parkinsonism, pugilistic encephalitis, Guam parkinsonism-dementia complex, Pick's disease, multiple system atrophy (MSA), progressive supranuclear paralysis (PSP) and corticobasal degeneration (CBD), Down syndrome, Lewy body disease, Huntington's disease, amyotrophic lateral sclerosis, multiple sclerosis, kuru, Gerstmann-Sträussler-Scheinker disease, senile cardiac amyloidosis, familial amyloid polyneuropathy, or amyloidosis associated with endocrine tumors such as medullary thyroid carcinoma.

Method for the Identification of a Compound Capable of Inhibiting Amyloid Deposit-Induced Cell Death

The inventors have observed that in cells of a neuronal origin, the addition of amyloid protein reproduces the pathological characteristics of a disease associated with amyloid deposits such as AD. Accordingly, those agents which bring about an improvement in pathological stages observed in a cell culture could be potentially useful in the treatment of diseases associated with amyloid deposits in general, and in the treatment of AD in particular.

Thus, in another aspect, the invention relates to a method for the identification of a compound capable of inhibiting amyloid deposit-induced cell death and/or suitable for the treatment of a disease associated with the formation of amyloid deposits, which method comprises:
a) contacting a first sample of a cell population with the amyloid protein and with a candidate compound and a second sample of said cell population with the amyloid protein and with a polypeptide comprising sequence SEQ ID NO:1 or a functionally equivalent variant thereof; and
b) determining in the cell populations of the first and second samples the level of at least one marker associated with amyloid deposit-induced cell death,

wherein if the level of at least one marker associated with amyloid deposit-induced cell death in the first sample is lower than the level of said marker in the second sample, it is indicative of the candidate compound being capable of inhibiting amyloid deposit-induced cell death and/or being useful for the treatment of a disease associated with the formation of amyloid deposits.

The terms “diseases associated with the formation of amyloid deposits,” “amyloid protein,” or “beta amyloid peptide,” “beta amyloid peptide-induced cell death” and “diseases associated with the formation of amyloid deposits” have been defined above and are applied in the same manner to the method of identifying compounds according to the invention.

One skilled in the art understands that in order to determine if a protein is an amyloid protein, said protein or peptide must appear in the amyloid deposits of diseases associated with amyloid deposits mentioned above, therefore having an elongated morphology; they are stained with Congo Red, exhibiting a characteristic X-ray diffraction pattern called a “cross-beta” pattern. Inouye H. et al. Biophys J. 1993 64(2):502-519.

In a particular embodiment, the amyloid protein is a beta amyloid peptide; accordingly, the method would be a method for the identification of compounds capable of inhibiting beta amyloid peptide-induced cell death for the treatment of diseases associated with the formation of beta amyloid peptide deposits. In a particular embodiment, the amyloid peptide is Aβ peptide (1-42), in which case the method would be a method for the identification of compounds capable of inhibiting beta amyloid peptide-induced cell death for the treatment of diseases associated with the formation of Aβ peptide (1-42) deposits.

Thus, in a first step (step a), the method for the identification of compounds capable of inhibiting amyloid deposit-induced cell death and/or suitable for the treatment of a disease associated with the formation of amyloid deposits comprises contacting a cell or a sample of a cell population with an amyloid protein.

In the present invention, cell is understood to mean a cell isolated in culture as well as a plurality of cells or cell population that are both isolated (cell culture) and part of an organotypic culture. Thus, in the present invention, the term “culture” or “cell population” includes all the aforementioned possibilities.

Organotypic cultures can be taken from any organ in which the influence of amyloid protein-induced, or particularly the Aβ peptide-induced, or more specifically Aβ peptide (1-42)-induced cell death is intended to be studied, such as, for example, in the brain, the heart, the kidneys, etc. In a particular embodiment, the organotypic culture is of cerebral origin. The organotypic culture may contain various areas of the brain and will be performed by selecting the areas according to the particular interests of each assay.

Methods for generating organotypic cultures are known to one skilled in the art (Gahwiler, 1981 J. Neurosci. Meth. 4, 329-42; Gahwiler 1984 Neuroscience. 11:751-60; Gahwiler 1988 Trends Neurosc. 11:484-9; Stoppini et al. 1991 J. Neuroscience Methods 37:173-82). The non-human animal whose brain or other organ has been dissected may be any animal, preferably, a vertebrate, such as a mammal, for example, a rodent, preferably, a mouse or a rat. Said non-human animal can be an animal with a genetically modified gene pool, i.e., that animal whose genetic material has been manipulated and designed or altered deliberately for the purpose of conferring to it a characteristic of interest, or it may be a non-genetically modified animal. Said genetically modified non-human animals can be transgenic animals, i.e., animals having, inserted in their genome, the sequence of a gene of interest, e.g., EYFP gene (Winter S M et al., 2007 Respir Physiol Neurobiol. 159:108-14), or GFP gene for the expression of the fluorescent protein GFP under control of the nestin promoter (Friling et al., 2009 Proc Natl Acad Sci USA 106(18):7613-8). They may also be an animal whose expression of a specific gene is blocked (e.g., knock-out mice). The methods for generating animals of this type are well known to one skilled in the art.

The cultures used in the method of the invention are kept under conditions that allow cell survival. Said conditions suitable for the survival of said culture include conditions which assure that the culture does necrotize and is kept alive. Said conditions include keeping the cultures under suitable conditions of humidity, temperature, and gas concentrations (usually 37° C., 5% CO₂, and 95% O₂), which conditions may be achieved by keeping the cultures in incubators especially designed for said purpose. The state of the art includes a number of examples of incubators suitable for keeping an organotypic culture alive. Culture conditions vary greatly for each type of organotypic culture.

In addition to the temperature and the mixture of gases, the factor which most commonly varies in culture systems is the growth medium. Growth medium recipes may vary in pH, glucose concentration, growth factors, and the presence of other nutritive components. Various recipes of media used for organotypic culture maintenance are known (Vergni et al., 2009 PLoS ONE4(4):e5278, Chechneva et al., 2006 Neurobiol of Dis. 23(2):247-59).

Furthermore, it is necessary to keep the cultures under conditions of sterility by using suitable methods, for example, sterilization, etc., as well as by handling the culture under conditions of sterility. The objective of all this is to prevent microbial contamination (caused by e.g. bacteria, yeasts, mycoplasmas, etc.) which would compete with the cells from the part of the brain for nutrients and/or could infect and eliminate said cells. In a particular embodiment, all manipulation is typically carried out in a laminar flow hood to prevent the entry of contaminating microorganisms. Antibiotics can also be added to the culture medium.

Likewise, for the correct survival of the culture it may, on occasion, be necessary to make changes to the culture medium on a regular basis; thus, in a particular embodiment, changes are made to the culture medium every 3 or 4 days.

As it is used herein, the expression “contacting” refers to the process whereby an amyloid protein, such as the Aβ peptide for example, comes into contact with a cell or organotypic culture, and includes any possible “in vitro” form of contacting an amyloid protein in an extracellular manner, as well as any method which allows an amyloid protein to be introduced into cells that are isolated or part of an organotypic culture.

Amyloid proteins can be obtained commercially or be produced using chemical or biological synthesis.

Concentrations of Aβ peptide, especially of Aβ peptide (1-42) used in the method of the invention, in the case of using cell cultures, range from between 0.001 μM and 40 μM, 0.05 μM and 30 μM, preferably between 0.15 μM and 10 μM, 0.3 and 5 μM, 0.1 μM and 2 μM. In a preferred embodiment, the culture is a cell culture and the concentration of Aβ peptide is between 0.5 μM and 3 μM and preferably 1.25 μM. In another particular embodiment, the culture is an organotypic culture and the concentration of the Aβ peptide is between 10 nM and 1 μM, preferably 100 nM.

In a particular embodiment, the amyloid protein contacted with the cell population is the beta amyloid Aβ peptide (1-42).

The compounds used in the tracking method can be both organic and inorganic chemical compounds. Among the organic compounds, said compound can be a biological polymer such as a nucleic acid or a protein.

In a particular embodiment, the compound to be tested is not isolated but rather is part of a more or less complex mixture, either derived from a natural source or being part of a library of compounds. Examples of libraries of compounds that can be tested according to the method of the present invention include, without limitation, libraries of peptides formed both by peptides and by peptide analogs comprising D-amino acids or peptides comprising non-peptide bonds, libraries of nucleic acids formed by nucleic acids with non-phosphodiester bonds of the phosphorothioate type or peptide nucleic acids, libraries of antibodies, of carbohydrates, of low molecular weight compounds, preferably organic molecules, of peptidomimetics, and the like. In the event of using a library of low molecular weight organic compounds, the library may have been preselected to contain compounds that can more readily access the interior of the cell. Thus, the compounds can be selected based on specific parameters such as size, lipophilicity, hydrophilicity, the capacity to form hydrogen bonds. If the candidate compound is part of a mixture having more or less complexity, the invention additionally comprises one or more steps of fractionating said mixture and repeating the method of the invention a variable number of times until the compound of the mixture responsible for the separation of elements forming the first complex of the invention is isolated. Methods for fractionating compounds present in a mixture include chromatography (thin layer chromatography, gas or size exclusion chromatography in gel, affinity chromatography), crystallization, distillation, filtration, precipitation, sublimation, extraction, evaporation, centrifugation, mass spectrometry, adsorption, and the like.

Alternatively, the compounds to be tested may be part of an extract obtained from a natural source. The natural source may be an animal or plant source and be obtained from any environment, including, without limitation, extracts from organisms found on land, in the air, in the sea, and the like.

One skilled in the art understands that for carrying out an assay “in vitro,” peptides isolated from fraction lysates or from whole cells derived, without limitation, from primary cells, transformed cells, cell lines, recombinants, bacteria, etc., may be used.

Incubation with the agent to be tested is performed at different concentrations and times of incubation. Moreover, the use of negative (without agent) and positive control reactions is advisable.

Step b) includes the determination of the levels of cell death or culture viability, or the markers associated with amyloid deposit-induced cell death. Methods for measuring the levels of cell death or culture or cell viability are known to one skilled in the art.

Illustrative non-limiting examples of such methods include visual inspection under a microscope using morphological criteria, such as structure conservation, the use of vital stains, the quantification of the expression of cell viability markers, the determination of apoptosis-induced cell death, etc.

Cell death may be determined using methods amply described in the state of the art, such as the incorporation of propidium iodide or annexin V labeling. Degenerating cells can be quantified by measuring the incorporation of said substances into the cell, as described in the methods section in the examples of the present invention. Another method that can be used is the measurement of mitochondrial activity, such as the MTT reduction assay, as described in the invention in the section entitled MTT reduction assay.

Another method is the quantification of the level of expression of a marker associated with cell death or with cell viability. Levels of expression of cell viability or cell death markers can be quantified using different methods that are well known in the state of the art. As it is used herein, the term “expression” refers to a process whereby a protein is produced from DNA. This process involves the transcription of the gene to a messenger RNA (mRNA) and the translation of this mRNA to a protein. The terms protein or polypeptide are used in an equivalent manner in the present invention. In the context of the invention, “changes in the levels of expression” of cell viability or cell death markers refers to any change in the production of the mRNA, of the protein, or of both, which produces altered relative levels of the mRNA, protein, or both, in a sample with respect to other molecules in the same sample. It can be seen that the levels of expression of a cell viability or cell death marker may be determined by means of determining the levels of mRNA in a sample or by means of determining the levels of the corresponding polypeptide. Alternatively, the polypeptide viability or death markers can be variants resulting from post-translational modifications, including fragments thereof.

The levels of expression of cell viability or cell death markers can be evaluated by means of any of a wide range of well-known methods for detecting the expression of a transcribed molecule (mRNA) or its corresponding protein. Methods for determining the transcribed molecule or protein are well known to one skilled in the art, such as quantitative PCR or using antibodies capable of binding to proteins encoded by said genes and subsequent quantification of the complexes formed by using, for example, techniques such as Western-blot, ELISA (enzyme-linked immunosorbent assay), RIA (radioimmunoassay), competitive EIA (competitive enzyme immunoassay), DAS-ELISA (double antibody sandwich ELISA), immunocytochemical and immunohistochemical techniques, techniques based on the use of protein biochips or microarrays, etc. Although virtually any cell viability or cell death marker may be used, in a particular embodiment, levels of phosphatidylserine on the outer face of the plasma membrane are determined. Under physiological conditions, the phospholipid phosphatidylserine is located on the inner face of the plasma membrane; when the apoptosis process begins, this phospholipid is positioned on the outer face of the plasma membrane and can be detected with annexin-V protein. This protein binds specifically to phosphatidylserine and since it is labeled with a fluorochrome, the phosphatidylserine-annexin V complex can be detected by flow cytometry.

In another particular embodiment of the invention, the cell viability or cell death may be determined using vital stains well known to one skilled in the art, for example, crystal violet. Various immunoassay and microscopy techniques described above may be used for detection thereof.

One skilled in the art understands that said methods for determining cell viability or cell death of the culture under study may be used independently of or combined with one another.

The term “lower level” refers to any level of expression of the amyloid deposit-induced cell death marker in a cell population lower than the value of expression of said marker in a second sample or reference value established as the level of expression of said marker in a cell population in the presence of amyloid peptide and the polypeptide comprising sequence SEQ ID NO: 1. Therefore, the levels of expression of the cell death marker are considered as being below or lower than their reference value when they are at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150% or higher, below their reference value.

The reference value is the level of expression of the cell death marker measured in the second sample which is obtained in a cell population that has been contacted with the amyloid protein and with a polypeptide comprising sequence SEQ ID NO: 1. Said value of expression corresponds to an average or mean level of the corresponding biomarker determined from a set of samples obtained from the same cell population in which the values of expression of said marker have been obtained.

The term marker associated with cell death refers to any molecule, such as proteins or enzymes, which are altered or overexpressed at the cellular level in cell death processes, whether they are caused by apoptosis, autophagy, or necrosis. The term furthermore includes the activation of enzymes associated with the cell death process or specific processes such as the production of reactive oxygen species or astrogliosis. Overexpression refers to the expression of an enzyme or protein that is greater than what is commonly observed in a cell or cell population. The term activation refers to the activity of this enzyme that is greater than what is commonly observed in a cell or cell population, or to a cellular process taking place with a higher intensity than what is observed in a cell or cell population.

Therefore, for identifying a compound capable of inhibiting amyloid deposit-induced cell death and/or suitable for the treatment of a disease associated with the formation of amyloid deposits, the level of a cell death marker will be measured in the first sample and compared with the level of said marker in the second sample, wherein if the level of the cell death marker in the first sample is less than the level of said marker in the second sample, it will mean that said compound is capable of inhibiting cell death or is suitable for the treatment of a disease associated with the formation of amyloid deposits. As indicated, the marker refers to the expression of proteins or enzymes, the activation of proteins or enzymes and/or cellular processes. Therefore, the method includes the determination of the level of protein or enzyme activity or the determination of products derived from processes which are overactivated in a cell or in a cell population in situations of cell death.

One skilled in the art understands that the level of expression, activity, or intensity of a process both in the first and in the second sample may be measured in absolute terms or in relation to the relative level of expression, activity, or intensity of said process in the absence of both the candidate compound and of the polypeptide comprising sequence SEQ ID NO:1 or a functionally equivalent variant thereof. That is, the relative level of expression, activity, or intensity of a process in the presence of the candidate compound will be measured in relation to the level of expression, activity, or intensity of a process in the absence of said candidate compound in the first sample, and the expression, activity, or intensity of a process in the presence of the polypeptide comprising sequence SEQ ID NO: 1 or a functionally equivalent variant will in turn be measured in relation to said level in the absence of the polypeptide comprising sequence SEQ ID NO: 1 or a functionally equivalent variant in the second sample. Therefore the final comparison between the level of expression, activity, or intensity of a process between the first and second samples will be performed by means of the comparison of the relative expression, relative activity, or relative intensity of a process in both samples.

In a particular embodiment, the polypeptide comprising sequence SEQ ID NO: 1 of the second sample is a polypeptide comprising sequence SEQ ID NO: 2, SEQ ID NO: 3, or SEQ ID NO: 4.

In another particular embodiment, the marker associated with the cellular response to amyloid peptide is selected from a group consisting of:
(i) Level of active Rac or ratio between active Rac and inactive Rac,
(ii) Level of NADPH oxidase (NOX) activity,
(iii) Level of reactive oxygen species,
(iv) Level of PKC activation,
(v) Level of GFAP expression,
(vi) Level of reactive astrogliosis in the dentate gyrus, and
(vii) Level of GRP78 expression in S100β-positive astrocytes.

A compound is potentially useful for the treatment of diseases associated with the formation of amyloid deposits such as, for example, diseases associated with Aβ or (1-42)Aβ deposits when the levels of Rac1 activation in the cell after having been treated with a candidate compound are less than before treatment. More specifically, a compound is potentially useful for the treatment of diseases associated with the formation of amyloid deposits when the levels of Rac1 activation in the cell after having been treated with a candidate compound are less than the levels of Rac1 activation in the second sample or reference sample as defined above, i.e., a cell population exposed to the amyloid peptide in the presence of the polypeptide containing the sequence SEQ ID NO: 1 or a functionally equivalent variant thereof.

Levels of Rac1 activation are considered to be less than the levels of the values of the reference sample when an activation that is lower by 2, 5, 10, 15, 20, 30, 40, 50, 100 times is produced in the cell or culture treated with the candidate compound in relation to the second sample or reference sample corresponding to a cell or culture treated with the polypeptide comprising sequence SEQ ID NO: 1 or a functionally equivalent variant thereof in the presence of amyloid deposits, such as the Aβ peptide, or more specifically the Aβ peptide (1-42).

One skilled in the art understands that the level of Rac1 activation both in the first and in the second sample may be measured in relation to the relative level of Rac1 activation in the absence of both the candidate compound and the polypeptide comprising sequence SEQ ID NO:1 or a functionally equivalent variant thereof. That is, it will be measured as the relative level of Rac1 activation in the presence of the candidate compound in relation to the level of Rac1 activation in the absence of said candidate compound, and the relative Rac1 activation in the second sample will in turn be measured in relation to Rac1 activation in the absence of the polypeptide comprising sequence SEQ ID NO: 1 or a functionally equivalent variant. Therefore, the final comparison between the level of Rac1 activation between the first and second samples will be performed by means of the comparison of the relative Rac1 activation in both samples.

One skilled in the art understands that if the method of measuring the activity of Rac1 requires lysis of the cell (e.g. protein extraction, RNA extraction, etc.), then it would be necessary to use a second cell or culture which has not been treated with the compound under study or with the polypeptide comprising sequence SEQ ID NO: 1 in which Rac1 activation (naïve-type control culture) will be measured to calculate relative Rac1 activation.

In a preferred embodiment, Rac1 activation is determined by means of determining the amount of Rac1 that is associated with the p21-binding domain of p21-activated kinase, PAK, as described by Bernard et al. (J. Biol. Chem. 1999, 274:13198-204)

Likewise, a compound is also considered to be useful for the treatment of diseases associated with the formation of amyloid deposits, such as for example diseases associated with Aβ or (1-42)Aβ deposits, when it is capable of inhibiting NADPH oxidase (NOX) activity.

NADPH oxidase or NOX refers to an enzyme complex which oxidizes the reduced form of nicotinamide adenine dinucleotide phosphate (NADP), at the same time reducing molecular oxygen to superoxide. The invention contemplates analysis of the activity of any of the NOX isoforms, including NOX1, NOX2, NOX3, NOX4, NOX5, DUOX1, and DUOX2.

As it is used herein, “inhibiting NADPH oxidase activity” refers to the process which reduces NADPH oxidase activity in relation to NADPH oxidase activity which has not been subjected to said process. In this case, a candidate compound is considered to inhibit NADPH oxidase activity when it is capable of reducing the activity thereof in a cell or cell population in relation to the activity observed in a cell or cell population which has not been in contact with said compound. In another particular embodiment, a candidate compound is considered to inhibit NADPH oxidase activity when it is capable of reducing the activity thereof in a cell or cell population in relation to the activity observed in a cell or cell population of the second sample, that is, it has been in contact with a polypeptide comprising sequence SEQ ID NO: 1 or a functionally equivalent variant. Therefore, a candidate compound is considered to be capable of inhibiting amyloid deposit-induced cell death and/or suitable for the treatment of a disease associated with the formation of amyloid deposits when it produces an inhibition of NADPH oxidase greater than the inhibition produced by the polypeptide comprising sequence SEQ ID NO: 1 in a cell or cell culture. The term “inhibition of NADPH oxidase greater than the inhibition produced by the polypeptide comprising sequence SEQ ID NO: 1 in a cell or cell culture” implies that the reduction in the activity in the sample treated with the candidate compound is at least 10%, 20%, 30%, 40%, 50%, 60%, 70%, 80%, 90%, 100%, 200%, 300%, 400%, 500%, 1000% with respect to that observed when using the polypeptide comprising sequence SEQ ID NO:1.

In a particular embodiment, the total NADPH oxidase activity of the first sample containing the candidate compound is compared with the total NADPH oxidase activity of the second sample containing the polypeptide comprising sequence SEQ ID NO: 1. In another particular embodiment, the relative NADPH oxidase activity of a cell or cell population in relation to NADPH oxidase activity in a cell or cell population not containing the candidate compound or the polypeptide comprising sequence SEQ ID NO: 1 is calculated. That is, the relative NADPH oxidase activity of the first sample containing the candidate compound in relation to NADPH oxidase activity in a cell or cell population not containing the candidate compound is calculated and compared with the relative activity of the second sample containing the polypeptide comprising sequence SEQ ID NO: 1 in relation to a cell or cell population not containing the polypeptide of sequence SEQ ID NO: 1.

As it is used herein, “inhibiting NADPH oxidase activity” furthermore refers to processes or methods which reduce or prevent NADPH oxidase over-activity in relation to NADPH oxidase not subjected to said process or method. “NADPH oxidase over-activity” refers to the activity of this enzyme which is greater than the activity commonly observed in a cell or cell population.

NADPH oxidase inhibition activity can be determined by detecting a decrease in the reactive oxygen species (ROS), whether it occurs outside or inside the cell, or by means of other methods known to those skilled in the art. Methods for determining NADPH oxidase activity based on the detection of ROS are widely known in the state of the art (see, for example, Cortés-Rios, Anal Biochem. 2017, 536:96-100).

An example of a method for measuring the amount of superoxide (O₂⁻) in cell cultures includes a method for measuring the reduction of the tetrazolium salt, WST-1, by superoxide dismutase (SOD). One skilled in the art will understand that there are other methods for measuring the production of superoxide in cell cultures.

Likewise, for the identification of a compound capable of inhibiting amyloid deposit-induced cell death and/or suitable for the treatment of a disease associated with the formation of amyloid deposits, the level of reactive oxygen species (ROS) may be measured. ROS are characterized by their high oxidizing capacity, where non-limiting examples of ROS include hydroxyl radicals, the superoxide anion, or hydrogen peroxide.

Thus, in the context of the method of the invention, a compound is considered to be capable of inhibiting amyloid deposit-induced cell death and/or suitable for the treatment of a disease associated with the formation of amyloid deposits when it is capable of reducing the formation of ROS in the first sample in comparison with the second sample.

As in the other cases, the absolute amount of ROS between the first and second samples, or the relative amount of both samples in relation to a cell or cell population not containing the candidate compound or the polypeptide comprising sequence SEQ ID NO: 1 or a functionally equivalent variant, may be compared.

Non-limiting examples of methodologies that may be used for the quantification of ROS include the use of fluorescent sensors readily oxidizable by the action of the ROS capable of detecting the oxidative activity in cells or tissues and giving rise to the corresponding oxidized derivatives, such as for example dihydroethidium, dihydrorhodamine, dichlorodihydrofluorescein, or dihydroxyphenoxazine. The level of ROS may also be measured by means of using aromatic boronates or other compounds known to one skilled in the art.

Likewise, according to the method of the invention a compound is considered to be capable of inhibiting amyloid deposit-induced cell death and/or suitable for the treatment of a disease associated with the formation of amyloid deposits when it is capable of inhibiting or reducing PKc activation to a greater extent than when the sample is treated with the polypeptide containing the sequence SEQ ID NO:1.

The term “inhibition of PKC activity greater than that produced by the polypeptide comprising sequence SEQ ID NO: 1 in a cell or cell culture” implies that the reduction in the activity in the sample treated with the candidate compound is at least 10%, 20%, 30%, 40%, 50%, 60%, 70%, 80%, 90%, 100%, 200%, 300%, 400%, 500%, 1000% with respect to that observed when using the polypeptide comprising sequence SEQ ID NO:1.

The kinase activity inhibitory capacity of a PKC inhibitor may be determined/measured using different assays measuring the kinase activity of PKCs. There are multiple methods which can be used for determining kinase, particularly of PKC activation known to one skilled in the art. By way of illustration, the capability of kinase to phosphorylate its natural substrate may be used (for example, proteins known to be activated by PKCs include proteins MARCKS, MAP kinase, PKD, IKB transcription factor inhibitor, VDR, Raf kinase, calpain, and EGFR). Methods based on measuring the activation of said proteins known to be activated by PKCs may also be used. The capability of a kinase to phosphorylate its substrate can be detected by any suitable method, for example, by means of radio/chemical/photochemical binding of a phosphate and the subsequent detection of its incorporation in the substrate.

Other PKC activation assays are designed for identifying the amount of active PKC. An example of a PKC activation assay comprises measuring by means of pull-down method, or a method based on the ELISA technique, where PKC activation is measured by means of luminescence. Techniques based on ELISA consist of incubating the assay with a plate having a PKC effector protein phosphorylation domain. The active form of PKC will bind to said domain and could subsequently be detected using a PKC-specific antibody.

Other assays for measuring kinase modulation are known to one skilled in the art, such as the assays described in document WO2004035811.

Likewise, the identification of a compound capable of inhibiting amyloid deposit-induced cell death and/or suitable for the treatment of a disease associated with the formation of amyloid deposits by means of quantifying the level of glial fibrillary acidic protein (GFAP). The present invention contemplates quantifying the level of GFAP expression and/the expression of any of its isoforms. The term “decrease in GFAP expression greater than that produced by the polypeptide comprising sequence SEQ ID NO: 1 in a cell or cell culture” implies that the reduction in the expression in the sample treated with the candidate compound is at least 10%, 20%, 30%, 40%, 50%, 60%, 70%, 80%, 90%, 100%, 200%, 300%, 400%, 500%, 1000% with respect to that observed when using the polypeptide comprising sequence SEQ ID NO:1.

The level of GFAP expression or the expression any of its isoforms may be measured by any of the methods mentioned in the context of the method of the invention when referring to measuring the level of expression and/or by any method known in the state of the art.

Likewise, the identification of a compound capable of inhibiting amyloid deposit-induced cell death and/or suitable for the treatment of a disease associated with the formation of amyloid deposits can be performed by means of evaluating the level of reactive astrogliosis in cells of the dentate gyrus. The term “level of reactive astrogliosis in cells the dentate gyrus greater than that produced by the polypeptide comprising sequence SEQ ID NO: 1 in a cell or cell culture” implies that the reduction in the level of reactive astrogliosis in cells the dentate gyrus in the sample treated with the candidate compound is at least 10%, 20%, 30%, 40%, 50%, 60%, 70%, 80%, 90%, 100%, 200%, 300%, 400%, 500%, 1000% with respect to that observed when using the polypeptide comprising sequence SEQ ID NO:1. One skilled in the art will have the necessary knowledge to evaluate the level of reactive astrogliosis. Examples of methodologies suitable for evaluating the level of reactive astrogliosis can be found in Kim T W et al. (Mol Cells. 2011, 31:379-83).

Lastly, in another embodiment, the identification of a compound capable of inhibiting amyloid deposit-induced cell death and/or suitable for the treatment of a disease associated with the formation of amyloid deposits can be performed by means of the level of GRP78 expression in S100β-positive astrocytes.

The term “level of GRP78 expression in S100β-positive astrocytes greater than that produced by the polypeptide comprising sequence SEQ ID NO: 1 in a cell or cell culture” implies that the reduction in the level of GRP78 in said cell line in the sample treated with the candidate compound is at least 10%, 20%, 30%, 40%, 50%, 60%, 70%, 80%, 90%, 100%, 200%, 300%, 400%, 500%, 1000% with respect to that observed when using the polypeptide comprising sequence SEQ ID NO:1. The level of GRP78 expression or the expression of any of its isoforms may be measured by any of the methods mentioned in the context of the method of the invention when referring to measuring the level of expression and/or by any method known in the state of the art.

In a particular embodiment, the cell population of the method of the invention is an astrocyte population or culture.

The astrocytes according to the particular embodiment of the present invention may or may not be reactive. The term reactive astrocytes or astrogliosis refers to a set of changes in the astrocytes occurring as a response to all the forms of damage and disease of the SNC. The changes produced by reactive astrogliosis vary with the severity of the damage caused to the SNC throughout a series of progressive alterations in the molecular expression, progressive cellular hypertrophy, and scar proliferation and formation. The damages caused to neurons in the SNC by an infection, trauma, cerebral ischemia, a stroke, an autoimmune response, or a neurodegenerative disease may result in reactive astrocytes. Reactive astrocytes can express the fibrillary acidic protein (GFAP) and/or the calcium-binding protein beta, S100beta.

The astrocytes of the present invention may derive from primary cultures from animal models of Alzheimer's or from human tissue samples, as well as from induced pluripotent stem cells. Likewise, the astrocytes used in the method of the invention may present mutations in the PSEN1 gene or the presence of APOE4.

The PSEN1 gene, according to the present invention encodes the protein presenilin 1, a subunit of the gamma secretase enzyme, which has the function of facilitating proteolysis of a number of proteins in the cell. Presenilin 1 plays an important role in the degradation of the amyloid precursor protein (APP), and mutations in the PSEN1 gene give rise to the early development of Alzheimer's disease.

APOE4 encodes dysfunctional protein, APOE-ε4, involved in Alzheimer's disease. APOE4 is one of the isoforms of the apolipoprotein-encoding gene. APOE-ε4 has been involved in a higher sensitivity to contracting Alzheimer's disease. 40-65% of patients with Alzheimer's have at least one copy of allele 4, although there are other factors involved, since at least one third of patients with Alzheimer's are ApoE4-negative and some people have both alleles in a homozygous state and do not develop Alzheimer's disease.

In a more particular embodiment, the astrocytes are hippocampal astrocytes.

In another embodiment, the disease associated with the formation of amyloid deposits is Alzheimer's disease, dementia associated with Lewy bodies, with Down syndrome, with Guam dementia complex associated with parkinsonism, with hereditary cerebral hemorrhage with amyloidosis-Dutch type, β-amyloid angiopathy, and cerebral hemorrhage such as cerebral hemorrhage due to solitary cerebral amyloid angiopathy osteomyelitis, tuberculosis, familial Mediterranean fever, hereditary cerebral hemorrhage, rheumatoid arthritis, Crohn's disease, ankylosing spondylitis, prion infections, Creutzfeldt-Jacob disease, type II diabetes, Castleman disease, amyloidosis associated with multiple myeloma, Parkinson's disease, subacute sclerosing panencephalitis parkinsonism, post-encephalitic parkinsonism, pugilistic encephalitis, Guam parkinsonism-dementia complex, Pick's disease, multiple system atrophy (MSA), progressive supranuclear paralysis (PSP) and corticobasal degeneration (CBD), Down syndrome, Lewy body disease, Huntington's disease, amyotrophic lateral sclerosis, multiple sclerosis, kuru, Gerstmann-Sträussler-Scheinker disease, senile cardiac amyloidosis, amyloid family polyneuropathy, or amyloidosis associated with endocrine tumors such as medullary thyroid carcinoma.

The invention is described below by means of the following merely illustrative and non-limiting examples of the scope of the invention.

### EXAMPLES

Materials and Methods

The reagents used were acquired from the companies SIGMA-ALDRICH, Bio-Rad, GE Healthcare, Thermo Scientific and Molecular Probes.

Construction and Generation of Recombinant Peptides Rs, Rd, Rw and Rt.

The PCR amplification of DNA fragments was performed by means of a standard method. A mixture containing 200 μM of each of the four deoxyribonucleotide triphosphates (dATP, dCTP, dGTP, dTTP), 10 pmol of each of the primers (for each of the Rs, Rd, Rw, and Rt fragments) 2 mM MgCl₂, 0.5 u/μL Ampli Taq Gold polymerase (Applied Biosystem), and 10 ng template DNA (which is integrin β1 DNA) was prepared, and all of it is diluted in the reaction buffer supplied by the manufacturer. The reactions are carried out in the GeneAmp® PCR System 2700 thermocycler (Applied Biosystem). The cycles were started out at 95° C. for 30 seconds; followed by 35 amplification cycles. Each amplification cycle consisted of 95° C. for 30 seconds; followed by 30 seconds at 60° C. and another 30 seconds at 72° C. At the end of the 35 cycles, the samples were incubated for 10 minutes at 72° C.

The amplified DNA fragments were isolated and separated by electrophoresis with 1% agarose in TAE buffer (40 mM Tris Acetate, 2 mM Na₂EDTA, Sigma-Aldrich) and in the presence of ethidium bromide.

The bands of interest were cleaved from the gel with a sterile scalpel, being visualized through a UV light transilluminator (CHEMIDOC XRS (Bio-Rad)). Extraction of the DNA fragments from the gel was performed with the QlAquick® Gel extraction kit (QIAGEN) following the manufacturer's instructions. After washing, the DNA was eluted in a Tris buffer pH 8.5 with a low concentration of salts.

The PCR products were cloned into the pGEX4T3 vector (GE/Healthcare). To that end, both the PCR fragments and the vector were digested with restriction enzymes (PstI and EcoRI), and subsequently the ends of the open vector were dephosphorylated with alkaline phosphatase. Both the dephosphorylated vector and the digested PCR fragments were separated in an agarose gel and purified as previously indicated.

The PCR products and the vector were then ligated to one another, which was done in a 10 μL reaction volume made up of 5 μL of reaction buffer, 50 ng/μL pGEX4T3, 1 T4 DNA ligase unit, 2 μL of the PCR product of interest, and 1 μL of molecular biology grade H₂O. This mixture was incubated at 22° C. for 3 hours. Lastly, the ligation product was introduced in competent bacteria Escherichia coli XL1 Blue (Stratagene). Briefly, 2 μg of DNA and 50 μL of bacteria were added in polypropylene tubes. The mixture was incubated for 30 minutes in ice, followed by exposure to 42° C. for 45 seconds; the bacteria were subsequently incubated in ice for 2 minutes. 300 μL of S.O.C. medium (Sigma) were then added, and the bacteria were incubated under stirring at 220 r.p.m. for one hour at 37° C. Lastly, 60 μL of liquid bacterial culture were seeded on LB agar plates that contained the resistance antibiotic of the seeded plasmid (100 μg/mL of ampicillin) and incubated in the oven at 37° C. for 16-18 hours. At the end of this time, it was verified that the colonies presented the plasmid of interest and a positive colony was taken and grown in 50 mL of 2XYT culture medium (Sigma) in the presence of 100 μg/mL of ampicillin overnight in an agitator at 220 r.p.m. at 37° C. The next morning, the culture was diluted ten-fold in 2XYT medium in the presence of ampicillin and left to stabilize under stirring at 220 r.p.m. at 37° C. for one hour. At the end of this time, expression of the fusion proteins was induced with 1 mM IPTG (Sigma) for 4 hours under stirring. Lastly, the bacteria were concentrated by centrifugation at 4000 r.p.m. for 15 minutes at 4° C., and the supernatant was discarded. The bacterial precipitate was resuspended in 6 mL of lysis buffer (20% Sucrose (Sigma), 10% glycerol (Sigma), 50 mM Tris-HCI pH 8 (Sigma), 200 mM sodium metabisulfite Na₂O₅S₂ (Sigma), 2 mM MgCl₂ (Sigma), 2 mM DTT (Sigma), 1 mM PMSF (Sigma), 1 mg/mL Aprotein (Sigma), 1 mg/mL Leupeptin (Sigma)).

The bacterial suspension was sonicated during 4 rounds of sonication (Misonix Inc. Microson™ Ultrasonic Cell Disruptor XL Model DU-2000) at a wave amplitude of 5000 μm for 10 seconds each, being left to stand for 10 seconds between each round, with the sample being kept in ice at all times. The sonication product was centrifuged at 14,000 r.p.m., collecting the supernatant and dividing it up into aliquots to be stored frozen at −80° C. The supernatants were contacted with a Glutathione-Sepharose matrix (one for each protein) for fixing fusion proteins to said matrix and subsequently eluting the fixed proteins with reduced glutathione and dialyzing them. After dialysis, the solution was recovered and stored at −80° C. until use.

Preparation of Amyloid β Peptide 1-42 Oligomers.

Amyloid β peptide 1-42 oligomers were prepared as described by Dahlgren et al., 2002 (J. Biol. Chem., 277(35):32046-32053). Briefly, amyloid β peptide 1-42 was dissolved in 1 mM of hexafluoroisopropanol (Sigma-Aldrich) and distributed in aliquots in sterile Eppendorf tubes. By applying a vacuum in a speed vac, hexafluoroisopropanol was completely removed, leaving a film which is the peptide and was stored at −80° C. For the aggregation protocol, the peptide was first resuspended in anhydrous DMSO (Sigma-Aldrich) at a concentration of 5 mM to finally take the peptide to a final concentration of 100 μM in Hams F-12 (PromoCell) and incubate it at 4° C. for 24 h. At the end of this time, the preparation was centrifuged at 14000 g for 10 minutes at 4° C. to remove the insoluble aggregates and the supernatants containing amyloid β peptide 1-42 were transferred to new Eppendorf tubes and stored at 4° C. until use.

Astrocyte Cultures

Primary astrocyte cultures from the cerebral cortex of P0-P2 Sprague Dawley rats were prepared as described by McCarthy et al., (J. Cell Biol. 1980; 85:890-902): The cortical lobes were extracted and digested enzymatically with 400 μl of 2.5% trypsin and 40 μl of 0.5% deoxyribonuclease in HBSS (Hank's balanced salt solution, Sigma-Aldrich) for 15 minutes at 37° C. The enzymatic reaction was stopped by adding IMDM medium supplemented with 10% FBS (Gibco) and centrifuging at 1200 rpm for 6 minutes. The cell pellet was resuspended in 1 ml of the same solution and mechanically dissociated using needles with a section gauge between 21 and 23G. The resulting cell suspension was centrifuged at 1200 rpm for 6 minutes and the cells were seeded in 75 cm² culture flasks coated with 30 μg/ml of poly-D-Lysine.

In Vitro Binding Assay Between Recombinant Peptides and the Amyloid β Peptide 1-42.

In vitro binding experiments were performed as described by Zugaza et al., (J. Biol. Chem. 2002 277(47):45377-45392). The Glutathione-Sepharose matrices loaded with 500 ng of the fusion proteins (GST-Rs, GST-Td, GST-Rw, GST-Rw) and 500 ng GST0 (as a control) were incubated with 100 pmol of the amyloid β peptide 1-42 in binding buffer (50 nM Tris-HCl pH 7.5, 150 mM NaCl). The proteins were eluted from the matrix by adding Laemmli buffer, and they were separated by SDS-PAGE and analyzed by Western blot, with the immunoreactive bands being visualized with an amyloid β peptide 1-42-specific (anti-6E10) antibody, followed by treatment with ECL (General Electric Healthcare).

Rac GTPase Activation Assay

Rac affinity precipitation experiments were carried out using the fusion protein GST-Pak1 RBD, where RBD is the Pak1-Rac interaction domain. The determination of the state of Rac activation was carried out as follows: the astrocyte cultures were treated with the GST-Rs peptide or not treated for subsequently stimulating with the amyloid β peptide 1-42 or not. Next, the cells were washed with PBS and lysed as described by Maillet et al., (Nat. Cell Biol. 2003; 5(7):633-639). The lysates were centrifuged at 4° C. for 15 minutes at 14,500 r.p.m. and incubated for 1 hour at 4° C. with 50 μg of fusion protein previously coupled to Glutathione-Sepharose beads. The precipitated proteins were dissociated from the matrix in Laemmli buffer and analyzed by Western blot. The immunoreactive bands were visualized with a Rac1-specific antibody followed by treatment with ECL (General Electric Healthcare).

Determination of ROS Generation.

The fluorescent dye CM-H2DCFDA was used to quantify the reactive oxygen species (ROS) generated in cells stimulated in vitro. 1×10⁶ astrocytes were exposed to 5 μM of oligomeric amyloid β peptide 1-42 alone or together with the GST-Rs fusion protein and incubated with 10 μM CM-H2DCFDA for 30 minutes immediately post-stimulus. After three washes with PBS, fluorescence at an emission wavelength of 485 nm and 520 nm.

Administration of GST-Rs and GST0 by Intrahippocampal Route in Adult Mice.

Ten-week old male mice (C57BL6/J) were administered intrahippocampal injections in the right dentate gyrus (DG). For surgery, the animals were anesthetized with 0.3 ml of avertin. The mice were immobilized with a stereotaxic device and injected with the corresponding preparations: the first group (Aβ) was injected with 3 μl of oligomeric amyloid β peptide 1-42 at a concentration of 10 μM, the second group (Aβ+GST0) with 3 μl of oligomeric amyloid β peptide 1-42+0.45 μg/μl of GST0, and the third group (Aβ+Rs) with 3 μl of oligomeric amyloid β peptide 1-42+0.45 μg/μl of Rs peptide. The coordinates of the injections were Bregma −2.2 mm, lateral 1.5 mm, and Dv 2 mm. After injection and before withdrawing it, the needle was left at the application site for 5 minutes to prevent refluxes.

The mice are anesthetized with chloral hydrate and perfused with 30 ml of PBS followed by 30 ml of 4% paraformaldehyde in 0.4 M buffer phosphate. The brains were extracted and then fixed with the same fixing solution for 4 hours at room temperature, and next treated with 30% sucrose in 0.1 M PBS pH 7.5 at 4° C., and they were stored in a cryoprotectant solution (30% ethylene glycol, 30% glycerol, and 10% 0.4 M buffer phosphate in bidistilled water) at −20° C.

Processing the Tissue.

The brains of the mice were cut in a Leica VT1200S microtome (Leica Microsystems) to obtain coronal sections 40 μm thick. The slices were stored in PBS at 4° C. and impermeabilized and blocked with 0.1 M PBS pH 7.5, 10% NGS, 0.1% Triton X100 for one hour at room temperature. Next, the slices were incubated with primary antibody overnight at 4° C. under stirring. At the end of this time, they were washed three times with 0.1 M PBS pH 7.5, 0.1% Triton X100 and incubated with blocking solution which contained the fluorochrome-conjugated secondary antibody for 1 hour at room temperature. Lastly, the slices were washed three times with washing buffer and incubated with 4 μg/ml of DAPI, two additional washes were performed with washing buffer and they were mounted on glass slides with Fluoromount-G mounting medium (Southern Biotech).

DAB Staining.

The slices were washed in PBS and incubated under light stirring in 0.1 M PBS containing 3% H₂O₂ for 10 minutes at room temperature. Then, the slices were washed three times with PBS and incubated with slight stirring in blocking solution (PBS pH 7.5, 4% HS, 0.1% Triton X100) for 30 minutes at room temperature. Next, the slices were incubated with the corresponding specific primary antibodies in the same blocking buffer, overnight at 4° C., and washed three times with PBS and incubated with the secondary antibodies in blocking solution for 1 hour at room temperature. The slices were incubated with the ABC complex following the manufacturer's instructions (Vector Laboratories) for 1 hour at room temperature and washed three times with PBS.

Immunofluorescence of Astrocyte Cultures.

Double immunofluorescence labelling was carried out in primary astrocyte cultures. 8 DIV after treatment with GST0 or Rs in the presence of oligomeric amyloid β peptide 1-42, the cultures were fixed in methanol for 10 minutes at 4° C. and washed three times with PBS. The fixed cells were permeabilized with blocking solution (4% goat serum, 0.1% Triton X100 in PBS) for 1 hour at room temperature and incubated with the primary antibody, anti-GRP78 or anti-S100β, in blocking buffer overnight at 4° C. At the end of this time, the cells were washed with washing solution (0.1 M PBS pH 7.5, 0.1% Triton X100) and incubated with the fluorochrome-conjugated secondary antibody in blocking solution for 1 hour at room temperature. Subsequently, the cells were incubated with 4 μg/ml of DAPI for 10 minutes to stain the nuclei. And lastly, the cells were washed again two times and the slides were mounted on a glass support with Fluoromount-G mounting medium (SouthernBiotech).

Results

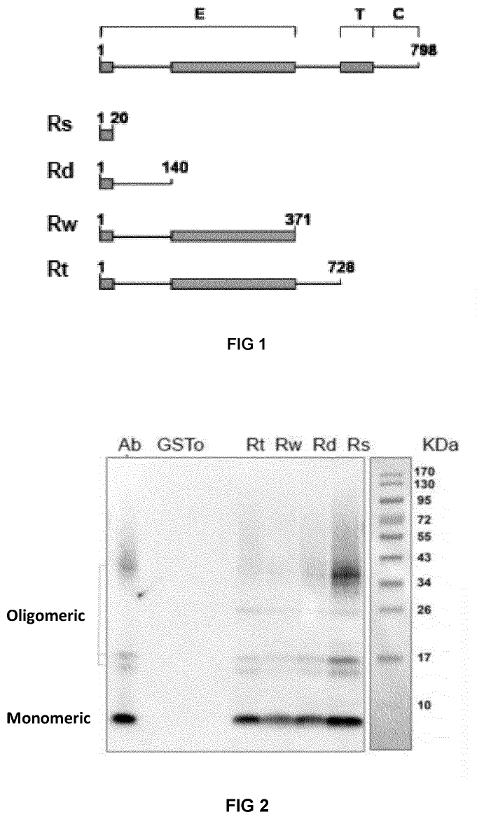

First, the extracellular amino acid sequence of integrin β1 was analyzed and divided into four regions for generating the mentioned peptides as shown in FIG. 1.

The first region, made up of the first 20 amino acids and known as signal peptide, was referred to as Rs; the second region from amino acid 1 to 139, Rd; the third region from amino acid 1 to 379 and including the VWA domain, Rw; and the final region including the entire extracellular domain amino acid 1 to 728, Rt.

For identifying the region or regions that may effectively interact with the oligomeric amyloid β peptide 1-42, the four GST fusion proteins with the different regions (Rs, Rd, Rw, Rt) were generated, and the oligomeric amyloid β peptide 1-42 binding capacity was examined by affinity chromatography in a Glutathione-Sepharose matrix.

As shown in FIG. 2, all the peptides were capable of binding not only to the monomeric form (intense band below 10 KDa), but also with greater efficacy to the oligomeric forms. The negative control, GST0, characterized by not having any additional peptide, was incapable of binding to amyloid β peptide 1-42. Therefore, these results indicate that the in vitro binding between the oligomeric amyloid β peptide 1-42 and the integrin β1 extracellular region is established by the signal peptide (Rs) of the latter.

To evaluate the effect of this GST-Rs peptide on the oligomeric amyloid β peptide 1-42-mediated Rac 1 GTPase activation in astrocytes, the cells were treated with 5 μM of oligomeric amyloid β peptide 1-42 with or without GST0 or GST-Rs for 60 minutes. Rac 1 activity was determined by affinity chromatography as indicated in the materials and methods section.

As was expected, the amyloid β peptide 1-42 mediated Rac1 activation, FIG. 3 (lane 2 compared to lane 1 of the first panel), the presence of GSTO did not hinder amyloid β peptide 1-42 from activating Rac1 (FIG. 3, lane 4 compared to lane 2), whereas the presence of Rs prevented this amyloid β peptide 1-42-mediated GTPase activation. The second panel of FIG. 3 represents total Rac 1.

Next, it was examined whether this GST-Rs peptide had any effect on the amyloid β peptide 1-42-mediated ROS generation in astrocytes as well.

As shown in FIG. 4, the amyloid β peptide 1-42 modulated Ros generation (histogram, blank bar), as occurred with Rac1 activation; in this case, GST0 did not interfere with the amyloid β peptide 1-42-mediated ROS generation (histogram, gray bar). However, the GST-Rs peptide was capable of conclusively blocking the amyloid β peptide 1-42-induced ROS generation. In summary, these results represented in FIGS. 3 and 4 demonstrate that GST-Rs peptide binds not only in vitro to amyloid β peptide 1-42, but furthermore, this recombinant peptide is functional at the cellular level since it is capable of preventing amyloid β peptide 1-42-induced intracellular signaling in primary astrocyte cultures.

It is widely accepted that the intracerebral administration of amyloid β peptide 1-42 leads to astrocyte reactivity in the dentate gyrus (DG) compared to controls in which a solvent is administered. The capability of this recombinant peptide, Rs, to prevent the amyloid β 1-42-mediated astrogliosis in the hippocampus was examined on this basis.

To quantify reactive astrogliosis, intrahippocampal injections of amyloid β peptide 1-42 (Aβ), Aβ with GST-Rs peptide (Aβ+GST-Rs), or Aβ with GST0 (Aβ+GST0) were carried out and the astrocyte markers (GFAP and S100β) were analyzed by means of immunostaining assays with DAB in different regions of the hippocampus. Gliosis was evaluated by analyzing the area occupied by GFAP and S100β in the DG and regions CA1 and CA3 of the hippocampus. As shown in FIGS. 5A and 5B, the GST-Rs peptide effectively reduced the presence of markers GFAP and S100β controlled by Aβ.

Immunohistochemical analysis showed a significant decrease in the values of GFAP (FIG. 5A) and S100β (FIG. 5B) in the presence of Aβ+GST-Rs compared with the ones obtained for those treated with Aβ (0.65±0.03 vs 0.94±0.07 for GFAP and 0.63±0.13 vs 0.93±0.13 for S100β). GST0 in the presence of Aβ did not prevent any change in GFAP (FIG. 5C) or in S100β (FIG. 5D) compared with the treatment of Aβ (1.31±0.09 vs 1.05±0.07 for GFAP and 1.48±0.16 vs 1.03±0.1 for S100β). These results demonstrate that the GST-Rs peptide prevents astrogliosis in the dentate gyrus of adult mice.

In addition to the area of the dentate gyrus, regions CA1 and CA3 were analyzed by immunohistochemistry, quantification of the area of GFAP and S100β expression (FIGS. 5A and 5B) demonstrated that neither region CA1 nor region CA3 present significant differences between the animals treated with Aβ or with Aβ+GST-Rs both in GFAP (FIG. 5A) and S100β (FIG. 5B). These results indicate that amyloid peptide 1-42-induced gliosis is specifically located in the dentate gyrus without there being any diffusion towards other regions of the hippocampus. Overall, these results show that recombinant protein Rs corresponding to the integrin β1 signal peptide effectively reduces in vivo astrogliosis in the dentate gyrus dependent on the amyloid β peptide 1-42.

At the astrocyte level, amyloid β peptide 1-42 also produced stress in the endoplasmic reticulum. GRP78 protein is a chaperone protein identified as an endoplasmic reticulum stress marker. Alberdi et al. (Aging Cell, 2013, 12:292-302) have described that acute injections of amyloid β peptide 1-42 in mouse brain induces GRP78 overexpression primarily in astrocytes. On this basis, it was postulated that the GST-Rs fusion protein could also prevent endoplasmic reticulum stress in astrocytes after the intracerebral injection of amyloid β peptide 1-42. To demonstrate this hypothesis, a double immunostaining assay for cerebral tissue S100β and GRP78 previously treated with Aβ, Aβ+GST-Rs, or Aβ+GST0 was carried out.

The intrahippocampal administration of the Rs peptide together with Aβ led to an unmitigated reduction of GRP78 expression in S100β-positive astrocytes (FIG. 6A) compared with Aβ alone. Moreover, FIG. 6B shows that GST0 protein in the presence of Aβ did not alter the result compared with the Aβ control alone. The quantification of the immunofluorescence staining demonstrates a significant decrease in the values of GRP87 in S100β-positive astrocytes (FIG. 6A) located in the dentate gyrus of the brains that were injected with Aβ+GST-Rs compared to the control (mice injected with Aβ (26.95±1.01 vs 30.64±1.24). Similarly to what is observed in FIG. 5, the GST0 control peptide did not produce any significant change in endoplasmic reticulum stress values in S100β-positive astrocytes (FIG. 6B) with respect to the control (mice injected with Aβ) (21.42±2.48 vs 22.07±1.36).

These results demonstrate that recombinant protein Rs corresponding to the integrin β1 signal peptide blocks amyloid β peptide 1-42-mediated endoplasmic reticulum stress.

These results show that a molecular tool with enormous potential has been developed for clarifying the molecular processes controlling the toxic program emanating from the amyloid β peptide 1-42/integrin β1 signaling pathway.

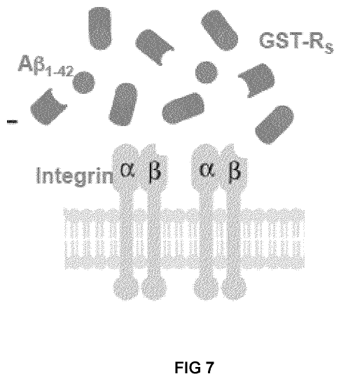

FIG. 7 depicts how the Rs peptide, and by extension Rd, Rw, and Rt peptides, function. These peptides bind in the signal region of integrin β1 (amino acid 1-20) to the amyloid β peptide 1-42 and thus prevent it from binding to integrin and activating its toxic program. These peptides have not only therapeutic potential but also can be used as a research laboratory reagent.

1. A method of treating and/or preventing a disease associated with the formation of amyloid deposits, which method comprises administering to a patient in need of such treatment an effective amount of a polypeptide comprising sequence SEQ ID NO: 1 or a functionally equivalent variant thereof.

2. The method of claim 1, wherein the polypeptide does not comprise the complete sequence of a β1 integrin.

3. The method of claim 1, wherein the polypeptide comprises sequence SEQ ID NO:2, SEQ ID NO:3, or SEQ ID NO:4.

4. The method of claim 1, wherein said disease associated with the formation of amyloid deposits is Alzheimer's disease, dementia associated with Lewy bodies, with Down syndrome, with Guam dementia complex associated with parkinsonism, with hereditary cerebral hemorrhage with amyloidosis-Dutch type, β-amyloid angiopathy, and cerebral hemorrhage such as cerebral hemorrhage due to solitary cerebral amyloid angiopathy osteomyelitis, tuberculosis, familial Mediterranean fever, hereditary cerebral hemorrhage, rheumatoid arthritis, Crohn's disease, ankylosing spondylitis, prion infections, Creutzfeldt-Jacob disease, type II diabetes, Castleman disease, amyloidosis associated with multiple myeloma, Parkinson's disease, subacute sclerosing panencephalitis parkinsonism, post-encephalitic parkinsonism, pugilistic encephalitis, Guam parkinsonism-dementia complex, Pick's disease, multiple system atrophy (MSA), progressive supranuclear paralysis (PSP) and corticobasal degeneration (CBD), Down syndrome, Lewy body disease, Huntington's disease, amyotrophic lateral sclerosis, multiple sclerosis, kuru, Gerstmann-Sträussler-Scheinker disease, senile cardiac amyloidosis, familial amyloid polyneuropathy, or amyloidosis associated with endocrine tumors such as medullary thyroid carcinoma.

5. The method of claim 4, wherein said disease is Alzheimer's disease.

6. A composition comprising a fusion protein comprising a polypeptide comprising sequence SEQ ID NO:1 or a functionally equivalent variant thereof and a compound suitable for the treatment of a disease associated with the formation of amyloid deposits.

7. The composition according to claim 6, wherein the polypeptide does not comprise the complete sequence of a β1 integrin.

8. The composition according to claim 6, wherein the polypeptide comprising sequence SEQ ID NO:1 is a polypeptide comprising sequence SEQ ID NO:2, SEQ ID NO:3, or SEQ ID NO:4.

9. The composition according to claim 6, wherein the compound suitable for the treatment of a disease associated with the formation of amyloid deposits is selected from the group consisting of:
(i) A cholinesterase inhibitor,
(ii) An NMDA receptor antagonist,
(iii) A beta amyloid peptide-specific antibody, and
(iv) A beta amyloid peptide aggregation inhibitor

10. The composition according to claim 9, wherein:
the cholinesterase inhibitor is selected from the group consisting of donepezil hydrochloride, rivastigmine, and galantamine,
(i) the NMDA receptor antagonist is memantine,
(ii) the beta amyloid peptide-specific antibody is selected from the group consisting of solanezumab, bapineuzumab, and gantenerumab,
(iii) the beta amyloid peptide aggregation inhibitor is selected from the group consisting of glycosaminoglycan 3-amino-1-propanesulfonic acid (3APS, tramiprosate), colostrinin, and scyllo-inositol.

11. (canceled)

12. A method of treating and/or preventing a disease associated with the formation of amyloid deposits which method comprises administering to a patient in need of such treatment an effective amount of the composition of claim 6.

13. The composition for use according to claim 12, wherein said disease associated with the formation of amyloid deposits is Alzheimer's disease, dementia associated with Lewy bodies, with Down syndrome, with Guam dementia complex associated with parkinsonism, with hereditary cerebral hemorrhage with amyloidosis-Dutch type, β-amyloid angiopathy, and cerebral hemorrhage such as cerebral hemorrhage due to solitary cerebral amyloid angiopathy osteomyelitis, tuberculosis, familial Mediterranean fever, hereditary cerebral hemorrhage, rheumatoid arthritis, Crohn's disease, ankylosing spondylitis, prion infections, Creutzfeldt-Jacob disease, type II diabetes, Castleman disease, amyloidosis associated with multiple myeloma, Parkinson's disease, subacute sclerosing panencephalitis parkinsonism, post-encephalitic parkinsonism, pugilistic encephalitis, Guam parkinsonism-dementia complex, Pick's disease, multiple system atrophy (MSA), progressive supranuclear paralysis (PSP) and corticobasal degeneration (CBD), Down syndrome, Lewy body disease, Huntington's disease, amyotrophic lateral sclerosis, multiple sclerosis, kuru, Gerstmann-Sträus sler-Scheinker disease, senile cardiac amyloidosis, familial amyloid polyneuropathy, or amyloidosis associated with endocrine tumors such as medullary thyroid carcinoma.

14. A method for the identification of a compound capable of inhibiting amyloid deposit-induced cell death and/or suitable for the treatment of a disease associated with the formation of amyloid deposits, which method comprises:
a) contacting a first sample of a cell population with the amyloid protein and with a candidate compound and a second sample of said cell population with the amyloid protein and with a polypeptide comprising sequence SEQ ID NO:1 or a functionally equivalent variant thereof; and
b) determining in the cell populations of the first and second samples the level of at least one marker associated with amyloid deposit-induced cell death,
wherein if the level of the at least one marker associated with amyloid deposit-induced cell death in the first sample is lower than the level of said marker in the second sample, it is indicative of the candidate compound being capable of inhibiting amyloid deposit-induced cell death and/or being useful for the treatment of a disease associated with the formation of amyloid deposits.

15. The method according to claim 14, wherein the beta amyloid peptide is Aβ peptide (1-42).

16. The method according to claim 14, wherein the polypeptide comprising sequence SEQ ID NO:1 is a polypeptide comprising sequence SEQ ID NO:2, SEQ ID NO:3, or SEQ ID NO:4 or wherein the polypeptide does not comprise the complete sequence of a β1 integrin.

17. The method according to claim 14, wherein the marker associated with the cellular response to an amyloid peptide is selected from the group consisting of
(i) Level of active Rac or ratio between active Rac and total Rac,
(ii) Level of NADPH oxidase (NOX) activity,
(iii) Level of reactive oxygen species,
(iv) Level of PKC activation,
(v) Level of GFAP expression,
(vi) Level of reactive astrogliosis in the dentate gyms, and
(vii) Level of GRP78 expression in S100β-positive astrocytes.

18. The method according to claim 14, wherein the cell population is an astrocyte population.

19. The method according to claim 18, wherein the astrocytes are hippocampal astrocytes.

20. The method according to claim 14, wherein said disease associated with the formation of amyloid deposits is Alzheimer's disease, dementia associated with Lewy bodies, with Down syndrome, with Guam dementia complex associated with parkinsonism, with hereditary cerebral hemorrhage with amyloidosis-Dutch type, β-amyloid angiopathy, and cerebral hemorrhage such as cerebral hemorrhage due to solitary cerebral amyloid angiopathy osteomyelitis, tuberculosis, familial Mediterranean fever, hereditary cerebral hemorrhage, rheumatoid arthritis, Crohn's disease, ankylosing spondylitis, prion infections, Creutzfeldt-Jacob disease, type II diabetes, Castleman disease, amyloidosis associated with multiple myeloma, Parkinson's disease, subacute sclerosing panencephalitis parkinsonism, post-encephalitic parkinsonism, pugilistic encephalitis, Guam parkinsonism-dementia complex, Pick's disease, multiple system atrophy (MSA), progressive supranuclear paralysis (PSP) and corticobasal degeneration (CBD), Down syndrome, Lewy body disease, Huntington's disease, amyotrophic lateral sclerosis, multiple sclerosis, kuru, Gerstmann-Sträus sler-Scheinker disease, senile cardiac amyloidosis, familial amyloid polyneuropathy, or amyloidosis associated with endocrine tumors such as medullary thyroid carcinoma.


SEQUENCE LISTING (45 sequences)

SEQ ID NO: 1 - protein, 20 aa, Artificial Sequence
  Human integrin beta 1 fragment
MNLQPIFWIGLISSVCCVFA

SEQ ID NO: 2 - protein, 140 aa, Artificial Sequence
  Human integrin beta 1 fragment
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAED

SEQ ID NO: 3 - protein, 371 aa, Artificial Sequence
  Human integrin beta 1 fragment
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLI

SEQ ID NO: 4 - protein, 728 aa, Artificial Sequence
  Human integrin beta 1 fragment
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPD

SEQ ID NO: 5 - protein, 16 aa, Artificial Sequence
  Cell penetrating peptide
RQIKIWFQNRRMKWKK

SEQ ID NO: 6 - protein, 34 aa, Artificial Sequence
  Cell penetrating peptide
DAATATRGRSAASRPTERPRAPARSASRPRRPVE

SEQ ID NO: 7 - protein, 24 aa, Artificial Sequence
  Cell penetrating peptide
RRIRPRPPRLPRPRPRPLPFPRPG

SEQ ID NO: 8 - protein, 9 aa, Artificial Sequence
  Cell penetrating peptide
RKKRRQRRR

SEQ ID NO: 9 - protein, 13 aa, Artificial Sequence
  Cell penetrating peptide
GRKKRRQRRRTPQ

SEQ ID NO: 10 - protein, 11 aa, Artificial Sequence
  Cell penetrating peptide
YGRKKRRQRRR

SEQ ID NO: 11 - protein, 20 aa, Artificial Sequence
  Cell penetrating peptide
ALWKTLLKKVLKAPKKKRKV

SEQ ID NO: 12 - protein, 15 aa, Artificial Sequence
  Cell penetrating peptide
RQIKWFQNRRMKWKK

SEQ ID NO: 13 - protein, 18 aa, Artificial Sequence
  Cell penetrating peptide
RGGRLSYSRRRFSTSTGR

SEQ ID NO: 14 - protein, 10 aa, Artificial Sequence
  Cell penetrating peptide
RRLSYSRRRF

SEQ ID NO: 15 - protein, 12 aa, Artificial Sequence
  Cell penetrating peptide
PIRRRKKLRRLK

SEQ ID NO: 16 - protein, 12 aa, Artificial Sequence
  Cell penetrating peptide
RRQRRTSKLMKR

SEQ ID NO: 17 - protein, 15 aa, Artificial Sequence
  Cell penetrating peptide
RRRRNRTRRNRRRVR

SEQ ID NO: 18 - protein, 19 aa, Artificial Sequence
  Cell penetrating peptide
KMTRAQRRAAARRNRWTAR

SEQ ID NO: 19 - protein, 13 aa, Artificial Sequence
  Cell penetrating peptide
TRRQRTRRARRNR

SEQ ID NO: 20 - protein, 13 aa, Artificial Sequence
  Cell penetrating peptide
GRKKRRQRRRPPQ

SEQ ID NO: 21 - protein, 13 aa, Artificial Sequence
  Cell penetrating peptide
GRRRRRRRRRPPQ

SEQ ID NO: 22 - protein, 17 aa, Artificial Sequence
  Cell penetrating peptide
KLALKLALKLALALKLA

SEQ ID NO: 23 - protein, 27 aa, Artificial Sequence
  Cell penetrating peptide
MGLGLHLLVLAAALQGAWSQPKKKRKV

SEQ ID NO: 24 - protein, 27 aa, Artificial Sequence
  Cell penetrating peptide
GALFLGWLGAAGSTMGAWSQPKKKRKV

SEQ ID NO: 25 - protein, 27 aa, Artificial Sequence
  Cell penetrating peptide
GALFLGFLGAAGSTMGAWSQPKKKRKV

SEQ ID NO: 26 - protein, 27 aa, Artificial Sequence
  Cell penetrating peptide
GALFLGFLGAAGSTMGAWSQPKSKRKV

SEQ ID NO: 27 - protein, 21 aa, Artificial Sequence
  Cell penetrating peptide
KETWWETWWTEWSQPKKKRKV

SEQ ID NO: 28 - protein, 21 aa, Artificial Sequence
  Cell penetrating peptide
KETWFETWFTEWSQPKKKRKV

SEQ ID NO: 29 - protein, 10 aa, Artificial Sequence
  Cell penetrating peptide
GRKKRRQRRR

SEQ ID NO: 30 - protein, 8 aa, Artificial Sequence
  Cell penetrating peptide
RRRRRRLR

SEQ ID NO: 31 - protein, 12 aa, Artificial Sequence
  Cell penetrating peptide
RRQRRTSKLMKR

SEQ ID NO: 32 - protein, 27 aa, Artificial Sequence
  Cell penetrating peptide
GWTLNSAGILLGKINLKALAALAKKIL

SEQ ID NO: 33 - protein, 33 aa, Artificial Sequence
  Cell penetrating peptide
KALAWEAKLAKALAKALAKHLAKALAKALKCEA

SEQ ID NO: 34 - protein, 16 aa, Artificial Sequence
  Cell penetrating peptide
RQIKIWFQNRRMKWKK

SEQ ID NO: 35 - protein, 11 aa, Artificial Sequence
  Cell penetrating peptide
YGRKKRRQRRR

SEQ ID NO: 36 - protein, 8 aa, Artificial Sequence
  Cell penetrating peptide
RKKRRQRR

SEQ ID NO: 37 - protein, 11 aa, Artificial Sequence
  Cell penetrating peptide
YARAAARQARA

SEQ ID NO: 38 - protein, 11 aa, Artificial Sequence
  Cell penetrating peptide
THRLPRRRRRR

SEQ ID NO: 39 - protein, 11 aa, Artificial Sequence
  Cell penetrating peptide
GGRRARRRRRR

SEQ ID NO: 40 - protein, 8 aa, Artificial Sequence
  Purification tag
DYKDDDDK

SEQ ID NO: 41 - protein, 8 aa, Artificial Sequence
  Purification tag
WSHPQFEK

SEQ ID NO: 42 - protein, 9 aa, Artificial Sequence
  Purification tag
YPYDVPDYA

SEQ ID NO: 43 - protein, 14 aa, Artificial Sequence
  Purification tag
GKPIPNPLLGLDST

SEQ ID NO: 44 - protein, 6 aa, Artificial Sequence
  Purification tag
AHGHRP

SEQ ID NO: 45 - protein, 21 aa, Artificial Sequence
  Purification tag
  misc_feature (19)..(20): Xaa can be any naturally occurring amino acid
PIHDHDHPHLVIHSGMTCXXC